(19) United States
(12) Patent Application Publication
(10) Pub. No.: US 2024/0409902 A1
(43) Pub. Date: Dec. 12, 2024

(54) SYSTEMS AND METHODS FOR CULTURING PODOCYTES AND USES THEREOF

(71) Applicant: Washington University, St. Louis, MO (US)

(72) Inventors: Hani Suleiman, St. Louis, MO (US); Guy Genin, St. Louis, MO (US); Jeffrey Miner, St. Louis, MO (US); Shumeng Jiang, St. Louis, MO (US); Chengqing Qu, St. Louis, MO (US)

(73) Assignee: Washington University, St. Louis, MO (US)

(21) Appl. No.: 18/705,835

(22) PCT Filed: Oct. 31, 2022
(86) PCT No.: PCT/US2022/048497

§ 371 Date: Apr. 29, 2024

Related U.S. Application Data
(60) Provisional application No. 63/273,627, filed on Oct. 29, 2021.

(51) Int. Cl. C12N 5/071

(52) U.S. Cl. CPC C12N 5/0686; C12N 2503/02; C12N 2533/52; C12N 2533/54

(57) ABSTRACT

Among the various aspects of the present disclosure is the provision of systems and methods for growing or culturing podocytes and uses thereof. In one aspect, a podocyte culture system including an ECM-patterned substrate is disclosed. The ECM-patterned substrate includes a compliant substrate. The compliant substrate includes a predetermined stiffness ranging from about 0.1 kPa to about 10 kPa. The ECM-patterned substrate further includes a plurality of patterned elements deposited over an exposed surface of the compliant substrate.

### CROSS-REFERENCE TO RELATED APPLICATIONS

This application claims priority from U.S. Provisional Application Ser. No. 63/273,627 filed on Oct. 29, 2021, which is incorporated herein by reference in its entirety.

### STATEMENT REGARDING FEDERALLY SPONSORED RESEARCH OR DEVELOPMENT

This invention was made with government support under DK058366 awarded by the National Institutes of Health and CMMI 1548571 awarded by the National Science Foundation (NSF). The government has certain rights in the invention.

### MATERIAL INCORPORATED-BY-REFERENCE

Not applicable

### FIELD

The present disclosure generally relates to methods and systems to culture podocytes.

### SUMMARY

Among the various aspects of the present disclosure is the provision of systems and methods for ex vivo culture of podocytes and uses thereof.

In one aspect, a podocyte culture system including an ECM-patterned substrate is disclosed. The ECM-patterned substrate includes a compliant substrate. The compliant substrate includes a predetermined stiffness ranging from about 0.1 kPa to about 10 kPa. The ECM-patterned substrate further includes a plurality of patterned elements deposited over an exposed surface of the compliant substrate. At least a portion of the patterned elements includes at least one extracellular matrix (ECM) protein. Each of the at least one ECM proteins is independently representative of a healthy or abnormal glomerular basement membrane (GBM). In some aspects, the compliant substrate includes a hydrogel. In some aspects, the hydrogel includes a polyacrylamide (PAAm) hydrogel or a hydroxyl PAAM hydrogel. In some aspects, the ECM-patterned substrate further includes a protein layer deposited over the exposed surface of the compliant substrate, wherein the plurality of patterned elements are deposited over the including at least one ECM protein. In some aspects, the at least one ECM protein is selected from a laminin, a collagen, a fibronectin, and any combination thereof. In some aspects, the at least one ECM protein is selected from a human laminin α5β2γ1 trimer (Lam-521), a fibronectin, a collagen IV, and any combination thereof, wherein the Lam-521 and the collagen IV are representative of healthy GBM; and the fibronectin is representative of abnormal GBM. In some aspects, the abnormal GBM includes a diseased GBM or an injured GBM. In some aspects, the ECM-patterned substrate includes the compliant substrate including a polyacrylamide (PAAm) hydrogel; and the plurality of patterned elements deposited over the exposed surface of the compliant substrate, where the plurality of patterned elements includes Lam-521. In some aspects, the ECM-patterned substrate includes the compliant substrate including a polyacrylamide (PAAm) hydrogel; the protein layer deposited over the exposed surface of the compliant substrate, where the protein layer includes collagen IV; and the plurality of patterned elements deposited over the exposed surface of the compliant substrate, where the plurality of patterned elements includes Lam-521; wherein the system is representative of healthy GBM. In some aspects, the system includes the compliant substrate including a polyacrylamide (PAAm) hydrogel; the protein layer deposited over the exposed surface of the compliant substrate, where the protein layer includes fibronectin; and the plurality of patterned elements deposited over the exposed surface of the compliant substrate, where the plurality of patterned elements includes Lam-521; where the system is representative of abnormal GBM. In some aspects, the predetermined stiffness of the compliant substrate ranges from about 0.2 kPa to about 0.9 kPa, representative of a physiological GBM stiffness. In some aspects, the predetermined stiffness of the compliant substrate ranges is about 6 kPa, representative of a pathological GBM stiffness. In some aspects, the pathological GBM stiffness is associated with hypertension or glycation. In some aspects, a spacing of adjacent patterned elements of the plurality of patterned elements ranges from about 1 micron to about 20 microns. In some aspects, the system includes a plurality of the ECM-patterned substrates and a 96 well-plate, wherein each ECM-patterned substrate is positioned in a well of the 96 well-plate.

In another aspect, a method of growing podocytes is disclosed that includes: providing a plurality of podocytes; providing the podocyte culture system described above; and seeding the podocytes onto the ECM-patterned substrate or adjacent to the ECM-patterned substrate. In some aspects, providing the plurality of podocytes includes isolating the plurality of podocytes from a biological sample. In some aspects, the biological sample is selected from a urine sample, a kidney biopsy sample, and a glomerular tissue sample.

In an additional aspect, a method of screening a candidate therapeutic agent is disclosed that includes: providing a plurality of podocytes; providing the podocyte culture system described above; culturing the plurality of podocytes using the podocyte culture system; contacting the plurality of podocytes with the candidate therapeutic agent; and observing an effect of the candidate therapeutic on correcting cellular homeostasis. In some aspects, the plurality of podocytes includes podocytes of a selected patient and the method is a patient-specific method. In some aspects, providing the plurality of podocytes includes isolating the plurality of podocytes from a biological sample or growing the podocytes out of iPSC-derived kidney organoids. In some aspects, the biological sample is selected from a urine sample, a kidney biopsy sample, and a glomerular tissue sample. In some aspects, the method further includes detecting sarcomere-like structures (SLSs), synaptopodin in SLSs, or motor protein myosin IIA in SLSs as markers of health or injury of the plurality of podocytes. In some aspects, the method further includes observing the growth of the plurality of podocytes or the markers of health or injury of the plurality of podocytes in response to the candidate therapeutic agent. In some aspects, the markers of health or disease of the plurality of podocytes include migration or biological markers selected from SLSs, synaptopodin, α-actinin 4, myosin IIA, and any combination thereof. In some aspects, the plurality of podocytes is human podocytes. In some aspects, the plurality of podocytes is obtained or previously obtained from a healthy subject. In some aspects, the plurality of podocytes are obtained or previously obtained from a subject having, suspected of having, or at risk for having a kidney disease or a glomerular disease. In some aspects, observing the effect of the candidate therapeutic on correcting cellular homeostasis further includes observing or identifying changes in podocyte cellular and cytoskeletal patterns or observing changes in cellular homeostasis. In some aspects, the plurality of podocytes cultured using the podocyte culture system upregulate sarcomere-like structures. In some aspects, the ECM-patterned substrate is further coated or printed with an antibody to a membrane protein, or to a binding partner of a membrane ligand. In some aspects, the ECM-patterned substrate is further coated or printed with an antibody to a membrane protein including nephrin. In some aspects, the compliant substrate is shaped on its surface using molding or reverse molding to form geometric features in the exposed surface prior to seeding with the plurality of podocytes.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a schematic of microprinting protocols for the fabrication of micropatterns with the desired ECM1 on a plain hydrogel substrate using lithographic printing methods.

FIG. 1B is a pair of images showing laminin 521 (left) or fibronectin (right) patterned on hydroxy PAAm hydrogel substrates using the protocol of FIG. 1A. Scale bars, 50 μm.

FIG. 1C is a schematic of the fabrication of micropatterns with the desired ECM1 using lithographic printing methods on a hydrogel substrate precoated with a desired ECM2.

FIG. 1D is a pair of images of patterned hydrogels with laminin 521 patterned upon fibronectin (left) or collagen IV (right) precoated over a hydroxyl PAAm hydrogel substrate produced using the protocol of FIG. 1C. The precoated hydrogel with the desired ECM2 enabled a coating both on and outside of the patterned area. Scale bars, 20 μm.

FIG. 2A is a set of images of spreading primary podocytes from an isolated mouse glomerulus found in the patterned area where Lam-521 is presented (marked with dashed boxes). Scale bars, 10 μm

FIG. 2B is a set of images of a hydrogel precoated with Lam-521 and printed with a blank stamp that shows Lam-521 outside of the patterned area (marked with boxes). Spreading podocytes are found only on the Lam-521. Scale bars, 10 μm.

FIG. 2C is a set of images showing podocytes that spread along the micropatterned Lam-521 before sending out protrusions on the fibronectin-enriched area outside the micropatterns. Scale bars, 10 μm.

FIG. 2D is a set of images showing that primary podocytes populated the Lam-521 micropatterned area first and then spread onto the collagen IV-coated area. Scale bars, 10 μm.

FIG. 3A is a set of images showing that SLSs could be identified after 2 days of culture, with alternating synaptopodin (which colocalized with α-actinin 4) and myosin IIA along the fibers. Scale bars, 5 μm (overview) and 2 μm (zoom-in view)

FIG. 3B is a graph of fluorescence intensity mapping along the fibers' direction showing myosin IIA alternating with colocalized synaptopodin and α-actinin 4.

FIG. 3C is a set of images showing alternating α-actinin 4 and myosin are attenuated after 6 days of culture. Scale bars, 5 μm (overview) and 2 m (zoom-in view).

FIG. 3D is a graph of fluorescence intensity mapping, which confirms the attenuated pattern shown in FIG. 3C.

FIG. 3E is a set of 2 images of Foot process-like structures sent by the cells observed in cultured podocytes, which were positive for synaptopodin, and linked to the synaptopodin-positive band templated by the SLSs. Scale bars, 10 μm (overview) and 2 μm (zoom-in view).

FIG. 3F is an image showing SLSs found in human primary podocytes after 3 days of culture. Scale bars, 10 μm (overview) and 2 μm (zoomed inset view).

FIG. 3G contains histograms of sarcomeric spacing as represented by both synaptopodin (left) and myosin IIA (right) signals that show a uniform distribution of around 0.8 to 1 μm after 2 days of culture (top row). The spacing was perturbed after 6 days of culture (bottom row). n=2009, 2089, 2129, and 2331 for Synpo/day 2, Synpo/day 6, Myosin IIA/day 2, and Myosin IIA/day 6, respectively.

FIG. 3H is a set of representative images of spreading podocytes on micropatterned hydrogels immunostained for synaptopodin and myosin IIA showing the attenuation of SLSs over time in the primary podocytes (compare primary and reseeded to long-term). In contrast, undifferentiated (nondiff) and differentiated immortalized podocytes showed no clear SLS patterns. Scale bar, 5 μm.

FIG. 3I is a cell counting graph of spreading podocytes on micropatterned hydrogels immunostained for synaptopodin and myosin IIA showing the attenuation of SLSs over time in the primary podocytes (compare primary and reseeded to long-term). In contrast, undifferentiated (nondiff) and differentiated immortalized podocytes showed no clear SLS patterns.

FIG. 4A is a set of images of podocytes showing that uniformly distributed 1-μm-long SLSs elongated to 1.4 μm when the substrate stiffness was decreased from 6.0 to 0.2 kPa. In contrast, the extremely stiff glass substrate (right) shortened this spacing to 0.8 μm. n=25, 36, 31, and 28 for 0.2 kPa, 0.9 kPa, 6.0 kPa, and glass. Scale bars, 5 μm.

FIG. 4B is a graph showing that the uniformly distributed 1-μm-long SLSs elongated to 1.4 μm when the substrate stiffness was decreased from 6.0 to 0.2 kPa. In contrast, the extremely stiff glass substrate shortened this spacing to 0.8 μm. n=25, 36, 31, and 28 for 0.2 kPa, 0.9 kPa, 6.0 kPa, and glass.

FIG. 4C is a schematic diagram (upper left) of an experimental setup used to model cell contractility. Estimated contours of maximum principal Cauchy stress (right) on different substrates showed increased contractility on stiffer hydrogels. The graph (lower left) depicts predictions of the shear stress component σ_{zx} acting in the x direction on the XY plane, along the line in the y direction that is pictured. Scale bars, 5 μm.

FIG. 4D is a set of images showing Myosin inhibition by blebbistatin caused a marked loss of SLSs inside podocytes cultured on glass, Scale bars, 5 μm.

FIG. 4E is a set of images showing Myosin inhibition by blebbistatin caused a marked loss of SLSs inside podocytes cultured either on glass (top row) or on patterned hydro gels (bottom row). Scale bars, 5 μm.

FIG. 4F is a set of images showing that combining the effect of myosin inhibition with that of a softer substrate caused the loss of SLSs with a lower concentration of blebbistatin. Scale bars, 5 μm.

FIG. 4G is a graph that shows that for podocytes on the stiffest substrate, myosin inhibition led to wider sarcomeric spacing. n=31, 30, 28, and 26 for 6.0 kPa, 6.0 kPa (Blebb), glass, and glass (Blebb).

FIG. 4H is a set of images from a detachment assay of reseeded primary podocytes with SLSs (1 day after reseeding) that shows significantly less detachment compared to podocytes with attenuated SLSs (6 days after reseeding) for longer durations. Scale bars, 100 μm.

FIG. 4I is a graph from a detachment assay of reseeded primary podocytes with SLSs (1 day after reseeding) that shows significantly less detachment compared to podocytes with attenuated SLSs (6 days after reseeding) for longer durations. Scale bars, 100 μm.

FIG. 5A contains a pair of images of cells on 0.9 kPa substrates just before (left) and 15 min after (right) application of CytoD. Upon disruption of the actin cytoskeleton and inhibition of further actin polymerization using cytochalasin D (CytoD), most cells elongated along their axes, indicating that they were in a state of contraction.

FIG. 5B is an image with strain mapping based on fluorescent microbeads embedded within the 0.9 kPa hydrogel following 15 minutes of treatment with CytoD. The podocytes were contractile, and the background contour plot shows the peak eigenvalue of the Green-Lagrange strain tensor field associated with the movement of beads as cells released tension over the 15-minute treatment with CytoD. Strains shown take the post-treatment state as the reference configuration.

FIG. 5C is a pair of images of cells on 0.9 kPa substrates wherein additional strain mapping was performed following the elimination of the cells using the detergent sodium dodecyl sulfate (SDS) that verifies that cell contraction was predominantly associated with contractility in the actin cytoskeleton. The strains associated with this subsequent SDS treatment (right image) were very small compared to those associated with CytoD (left image).

FIG. 5D is a pair of images of strains associated with contraction on 6 kPa substrates transmitted through the actin cytoskeleton, calculated as in FIGS. 5B and 5C, which were smaller on the stiffer 6 kPa substrates as compared to the strains observed on the 0.9 kPa substrates shown in FIGS. 5B and 5C. Strains associated with CytoD treatment (left) were much larger than those associated with subsequent SDS treatment (right).

FIG. 5E is a graph summarizing cell lengths before and after CytoD treatment on 0.9 kPa substrates. Paired t-tests revealed that the increases in cell length associated with 15 min of CytoD treatment were statistically significant.

FIG. 5F is a graph of cell length before and after CytoD treatment on 6 kPa substrates. Less, but still significant cell relaxation was recorded on a hydrogel with a stiffness of 6 kPa (FIG. 5F) compared to 0.9 kPa. Scale bar, 20 μm.

FIG. 6 contains a set of images showing a Laminin 521 micropatterned substrate (top row) and a Fibronectin micropatterned substrate (bottom row) stained for glomerulus/podocyte (Synpo, left), pattern protein (Laminin 521/Fibronectin, center). The right-most images are combined images of glomerulus/podocyte and pattern protein for each pattern protein. Laminin 521 micropatterns, but not Fibronectin micropatterns, were efficient at enabling glomerulus/podocyte attachment on the hydrogels. Scale bars, 50 μm.

FIG. 7 contains a set of images showing Laminin 521 micropatterned on a Fibronectin substrate stained for pattern proteins (left) and podocytes (center). As shown in the combined image (right), the preferential adhesion/migration on laminin-521 is specific for podocytes, as there are many cells without synaptopodin attached to the fibronectin between the laminin-521 micropatterns. Scale bars, 10 μm.

FIG. 8 contains a set of images showing that when cultured on Laminin 521-micropatterned hydrogels with Fibronectin outside the micropatterns, the podocytes send out synaptopodin-positive protrusions (marked by arrows); these seem to enable migration to a nearby micropattern (bottom row). Scale bars, 20 μm.

FIG. 9 is a set of images that show podocytes with SLSs on day 2 (top row) that lose SLSs by day 6 (bottom row). Scale bars, 5 μm.

FIG. 10 is a set of images showing immunostaining for nephrin in spreading primary podocytes (right center), showing no significant accumulation of nephrin. Scale bars, 5 μm.

FIG. 11 is a set of images showing immunostaining for α-SMA (center right) in spreading primary podocytes that shows no signal in synaptopodin-positive cells with SLSs. However, α-SMA is present in non-podocyte cells identified by negative synaptopodin immunostaining. Scale bars, 10 μm.

FIG. 12 is a set of images showing synaptopodin (left), α-actinin4 (left center), and myosin IIA (center right) staining of spreading podocytes isolated from Synpo−/− mice. While no synaptopodin signal was present, podocytes still presented with striated SLS patterns identified as alternating α-actinin4 and myosin IIA staining. Scale bars, 5 μm.

FIG. 13 is a pair of graphs that show that in differentiated immortalized podocytes (bottom), the SLSs showed dramatically larger spacings between synaptopodin-positive bands compared to primary podocytes (top), whereas myosin showed multiple bands in between synaptopodin bands

FIG. 14 is a set of images that show primary podocytes spread on non-patterned soft (200 Pa, top row) and stiff (6000 Pa, bottom row) hydrogels coated with laminin-521, in which both showed SLS patterns. Scale bars, 10 μm.

FIG. 15 is a set of images that show 3T3 cells cultured on hydrogels of different stiffnesses that showed significantly more stress fibers on the 6000 Pa substrate (bottom row) as compared to the 200 Pa substrate (top row). Scale bars, 10 μm.

FIG. 16 contains a schematic and graphs similar to FIG. 4C demonstrating that simulated shear stress at the adhesion surface showed dramatically reduced shear stress when the contractility inside of the cells was inhibited.

FIG. 17 is a set of images that shows mild myosin inhibition was not enough to eliminate all the SLSs inside the spreading podocytes, but SLSs in the center of the cells were disturbed due to shear lag effects. Scale bars, 10 μm.

FIG. 18 is a set of images showing blebbistatin wash-out led to the regeneration of SLSs in podocytes within 2 hours (top row). Longer (1 day) recovery (bottom row) allowed restoration of the SLSs even at the center of the cells. Scale bars, 5 μm

FIG. 19 is a top view of an ECM-patterned substrate in accordance with one aspect of the disclosure.

FIG. 20 is a side view of an ECM-patterned substrate in accordance with one aspect of the disclosure.

### DETAILED DESCRIPTION

The present disclosure is based, at least in part, on the discovery that a physiologically relevant microenvironment for podocyte cells enables observation of how the cells would respond in the body. As described in the Examples below, podocytes were isolated from the glomeruli of mouse kidneys isolated using magnetic beads or other existing methods, and the isolated podocytes were grown on microprinted hydrogels configured to culture the podocytes in a physiologically relevant manner. The devices and methods described here provide physiologically representative podocytes suitable for a variety of applications including, but not limited to, basic research of podocyte function and physiology, drug screening and/or patient-specific drug screening for treatment or target discovery, and drug discovery for kidney, podocyte, or glomerular diseases, disorders, or conditions.

### Podocyte Culture System

In some aspects, a podocyte culture system is disclosed that includes a substrate patterned with one or more proteins including, but not limited to, extracellular matrix (ECM) proteins. Without being limited to any particular theory, the materials and architecture of the podocyte culture system are selected to provide culture conditions similar to the in vivo microenvironment of glomerular podocytes. In some aspects, the substrate material is selected to approximately match the stiffness of the glomerular basement membrane (GBM). As described in the Examples below, substrate stiffness influenced the development and morphology of cultured podocytes. In other aspects, one or more extracellular matrix (ECM) proteins representative of the GBM in health and/or disease/injury proteins are patterned on the substrate to approximately match the in vivo microenvironment of podocytes.

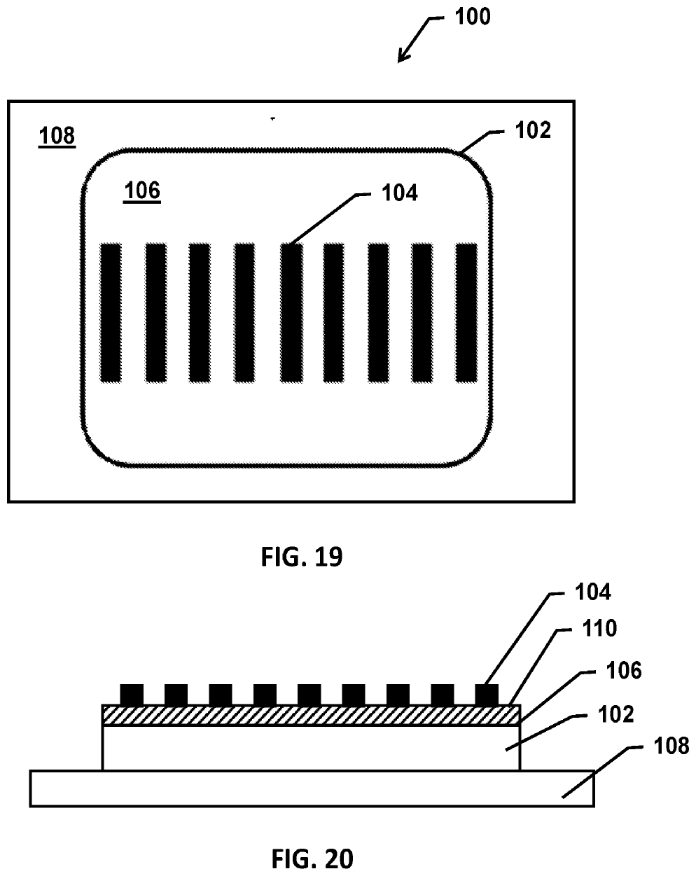

FIGS. 19 and 20 contain top and side views of an ECM-patterned substrate 100 in one aspect. In various aspects, the ECM-patterned substrate 100 includes a plurality of features selected to provide a culture environment that is representative of the physiological conditions experienced by podocytes in vivo. Referring to FIG. 19, the ECM-patterned substrate 100 includes a substrate 102 and a plurality of patterned ECM protein features 104 formed on an exposed surface 106 of the substrate 102. In some aspects, the substrate 102 may be formed on a support 108 selected to provide a rigid support surface for the substrate 102 to allow for moving, storing, and otherwise manipulating the ECM-patterned substrate 100 as needed during fabrication and use of the substrate 100 as described herein.

### Hydrogel Substrate

In some aspects, the substrate 102 is formed using a compliant material selected to approximate the material properties of a glomerular basement membrane to help provide an approximate match of in vivo conditions for the podocytes as described herein. In various aspects, the substrate may be produced with any suitable biocompatible material with suitable material properties without limitation. Non-limiting examples of suitable substrate materials are described in additional detail below. In one non-limiting example, the substrate may be produced using a hydrogel including, but not limited to, a hydroxyl PAAM gel.

In various aspects, the hydrogel substrate may be provided in any suitable shape without limitation. Non-limiting examples of suitable shapes include flat or planar, curved such that an exposed surface is concave or convex with at least one peak, and tubular. In some aspects, the exposed surface 106 of the substrate may further include any suitable surface texture without limitation. Non-limiting examples of suitable textures include smooth, rough, grooved, dimpled, speckled, or any other suitable texture.

As described herein, any hydrogel suitable for use in cell culture can be used. Furthermore, any hydrogel suitable for protein-coating patterning (e.g., ECM proteins) can be used. In particular, polyacrylamide hydrogels (e.g., hydroxyl PAAM) are suitable for use in protein coating or printing.

Methods of making and choosing appropriate hydrogel substrates are well known; see e.g. Ahmed 2015 J Adv Res. 6(2) 105-121; Calo 2015 Eur Poly J. 65 252-267. Except as otherwise noted herein, therefore, the process of the present disclosure can be carried out in accordance with such processes.

Compositions and methods as described herein provide for the use of polyacrylamide (PA) hydrogels. Components of PA hydrogels can comprise acrylamide, N,N′-methylenebisacrylamide (bis-acrylamide), and N-ethanal acrylamide monomers.

In some embodiments, a co-polymer such as an acrylamide can comprise about 1% to about 50% of the hydrogel by volume. Preferably, the co-polymer (e.g., acrylamide) can comprise about 4% to 15% of the hydrogel by volume. For example, the co-polymer (e.g., acrylamide) can comprise about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; or about 50% of the hydrogel by volume.

In some embodiments, a co-polymer crosslinker, such as bis-acrylamide can comprise between about 0.1% and about 5% of the hydrogel by volume. Preferably, the co-polymer or crosslinker can comprise between about 0.2% and about 1.2% of the hydrogel by volume. For example, the co-polymer or crosslinker can comprise about 0.1%; 0.2%; 0.3%; 0.4%; 0.5%; 0.6%; 0.7%; 0.8%; 0.9%; 1%; 1.1%; 1.2%; 1.3%; 1.4%; 1.5%; 1.6%; 1.7%; 1.8%; 1.9%; 2%; 2.1%; 2.2%; 2.3%; 2.4%; 2.5%; 2.6%; 2.7%; 2.8%; 2.9%; 3%; 3.1%; 3.2%; 3.3%; 3.4%; 3.5%; 3.6%; 3.7%; 3.8%; 3.9%; 4%; 4.1%; 4.2%; 4.3%; 4.4%; 4.5%; 4.6%; 4.7%; 4.8%; 4.9%; or 5% of the hydrogel by volume.

In some embodiments, an aldehyde-containing co-polymer or acrylamide monomer, such as N-ethanal acrylamide, can be between about 0.1% and about 10% of the hydrogel by volume. Preferably, the aldehyde-containing polymer or acrylamide can be 1.33% (1:75) of the hydrogel by volume. For example, the aldehyde-containing polymer or acrylamide can be about 0.1%; 0.2%; 0.3%; 0.4%; 0.5%; 0.6%; 0.7%; 0.8%; 0.9%; 1%; 1.1%; 1.2%; 1.3%; 1.4%; 1.5%; 1.6%; 1.7%; 1.8%; 1.9%; 2%; 2.1%; 2.2%; 2.3%; 2.4%; 2.5%; 2.6%; 2.7%; 2.8%; 2.9%; 3%; 3.1%; 3.2%; 3.3%; 3.4%; 3.5%; 3.6%; 3.7%; 3.8%; 3.9%; 4%; 4.1%; 4.2%; 4.3%; 4.4%; 4.5%; 4.6%; 4.7%; 4.8%; 4.9%; 5%; 5.1%; 5.2%; 5.3%; 5.4%; 5.5%; 5.6%; 5.7%; 5.8%; 5.9%; 6%; 6.1%; 6.2%; 6.3%; 6.4%; 6.5%; 6.6%; 6.7%; 6.8%; 6.9%; 7%; 7.1%; 7.2%; 7.3%; 7.4%; 7.5%; 7.6%; 7.7%; 7.8%; 7.9%; 8%; 8.1%; 8.2%; 8.3%; 8.4%; 8.5%; 8.6%; 8.7%; 8.8%; 8.9%; 9%; 9.1%; 9.2%; 9.3%; 9.4%; 9.5%; 9.6%; 9.7%; 9.8%; 9.9%; or 10% by volume.

In some embodiments, the components of the hydrogel can be varied in order to vary the hydrogel stiffness. For example, the hydrogel stiffness can vary from approximately 0.1 kilopascals (kPa) to approximately 200 kPa. In some aspects, the hydrogel stiffness varies from about 0.2 kPa to about 0.9 kPA, representative of a physiological GBM stiffness. In other aspects, the hydrogel stiffness is about 0.6 kPa, representative of a pathological GBM stiffness. stiffness. Preferably, the hydrogel stiffness can vary from about 0.1 kPa to about 100 kPa. For example, the hydrogel stiffness can be about 0.1 kPa; 0.2 kPa; 0.3 kPa; 0.4 kPa; 0.5 kPa; 0.6 kPa; 0.7 kPa; 0.8 kPa; 0.9 kPa; 1 kPa; 1.5 kPa; 2 kPa; 2.5 kPa; 3 kPa; 3.5 kPa; 4 kPa; 4.5 kPa; 5 kPa; 5.5 kPa; 6 kPa; 6.5 kPa; 7 kPa; 7.5 kPa; 8 kPa; 8.5 kPa; 9 kPa; 9.5 kPa; 10 kPa; 10.5 kPa; 11 kPa; 11.5 kPa; 12 kPa; 12.5 kPa; 13 kPa; 13.5 kPa; 14 kPa; 14.5 kPa; 15 kPa; 15.5 kPa; 16 kPa; 16.5 kPa; 17 kPa; 17.5 kPa; 18 kPa; 18.5 kPa; 19 kPa; 19.5 kPa; 20 kPa; 20.5 kPa; 21 kPa; 21.5 kPa; 22 kPa; 22.5 kPa; 23 kPa; 23.5 kPa; 24 kPa; 24.5 kPa; 25 kPa; 25.5 kPa; 26 kPa; 26.5 kPa; 27 kPa; 27.5 kPa; 28 kPa; 28.5 kPa; 29 kPa; 29.5 kPa; 30 kPa; 30.5 kPa; 31 kPa; 31.5 kPa; 32 kPa; 32.5 kPa; 33 kPa; 33.5 kPa; 34 kPa; 34.5 kPa; 35 kPa; 35.5 kPa; 36 kPa; 36.5 kPa; 37 kPa; 37.5 kPa; 38 kPa; 38.5 kPa; 39 kPa; 39.5 kPa; 40 kPa; 40.5 kPa; 41 kPa; 41.5 kPa; 42 kPa; 42.5 kPa; 43 kPa; 43.5 kPa; 44 kPa; 44.5 kPa; 45 kPa; 45.5 kPa; 46 kPa; 46.5 kPa; 47 kPa; 47.5 kPa; 48 kPa; 48.5 kPa; 49 kPa; 49.5 kPa; 50 kPa; 50.5 kPa; 51 kPa; 51.5 kPa; 52 kPa; 52.5 kPa; 53 kPa; 53.5 kPa; 54 kPa; 54.5 kPa; 55 kPa; 55.5 kPa; 56 kPa; 56.5 kPa; 57 kPa; 57.5 kPa; 58 kPa; 58.5 kPa; 59 kPa; 59.5 kPa; 60 kPa; 60.5 kPa; 61 kPa; 61.5 kPa; 62 kPa; 62.5 kPa; 63 kPa; 63.5 kPa; 64 kPa; 64.5 kPa; 65 kPa; 65.5 kPa; 66 kPa; 66.5 kPa; 67 kPa; 67.5 kPa; 68 kPa; 68.5 kPa; 69 kPa; 69.5 kPa; 70 kPa; 70.5 kPa; 71 kPa; 71.5 kPa; 72 kPa; 72.5 kPa; 73 kPa; 73.5 kPa; 74 kPa; 74.5 kPa; 75 kPa; 75.5 kPa; 76 kPa; 76.5 kPa; 77 kPa; 77.5 kPa; 78 kPa; 78.5 kPa; 79 kPa; 79.5 kPa; 80 kPa; 80.5 kPa; 81 kPa; 81.5 kPa; 82 kPa; 82.5 kPa; 83 kPa; 83.5 kPa; 84 kPa; 84.5 kPa; 85 kPa; 85.5 kPa; 86 kPa; 86.5 kPa; 87 kPa; 87.5 kPa; 88 kPa; 88.5 kPa; 89 kPa; 89.5 kPa; 90 kPa; 90.5 kPa; 91 kPa; 91.5 kPa; 92 kPa; 92.5 kPa; 93 kPa; 93.5 kPa; 94 kPa; 94.5 kPa; 95 kPa; 95.5 kPa; 96 kPa; 96.5 kPa; 97 kPa; 97.5 kPa; 98 kPa; 98.5 kPa; 99 kPa; 99.5 kPa; 100 kPa; 101 kPa; 102 kPa; 103 kPa; 104 kPa; 105 kPa; 106 kPa; 107 kPa; 108 kPa; 109 kPa; 110 kPa; 111 kPa; 112 kPa; 113 kPa; 114 kPa; 115 kPa; 116 kPa; 117 kPa; 118 kPa; 119 kPa; 120 kPa; 121 kPa; 122 kPa; 123 kPa; 124 kPa; 125 kPa; 126 kPa; 127 kPa; 128 kPa; 129 kPa; 130 kPa; 131 kPa; 132 kPa; 133 kPa; 134 kPa; 135 kPa; 136 kPa; 137 kPa; 138 kPa; 139 kPa; 140 kPa; 141 kPa; 142 kPa; 143 kPa; 144 kPa; 145 kPa; 146 kPa; 147 kPa; 148 kPa; 149 kPa; 150 kPa; 151 kPa; 152 kPa; 153 kPa; 154 kPa; 155 kPa; 156 kPa; 157 kPa; 158 kPa; 159 kPa; 160 kPa; 161 kPa; 162 kPa; 163 kPa; 164 kPa; 165 kPa; 166 kPa; 167 kPa; 168 kPa; 169 kPa; 170 kPa; 171 kPa; 172 kPa; 173 kPa; 174 kPa; 175 kPa; 176 kPa; 177 kPa; 178 kPa; 179 kPa; 180 kPa; 181 kPa; 182 kPa; 183 kPa; 184 kPa; 185 kPa; 186 kPa; 187 kPa; 188 kPa; 189 kPa; 190 kPa; 191 kPa; 192 kPa; 193 kPa; 194 kPa; 195 kPa; 196 kPa; 197 kPa; 198 kPa; 199 kPa; or 200 kPa.

In some embodiments, the temperature at which the hydrogel incubates can be varied to vary the hydrogel stiffness and ECM fiber length. Preferably, the incubation temperature can be between about 4° C. and about 37° C. For example, the temperature at which the hydrogel incubates can be about 1° C.; 2° C.; 3° C.; 4° C.; 5° C.; 6° C.; 7° C.; 8° C.; 9° C.; 10° C.; 11° C.; 12° C.; 13° C.; 14° C.; 15° C.; 16° C.; 17° C.; 18° C.; 19° C.; 20° C.; 21° C.; 22° C.; 23° C.; 24° C.; 25° C.; 26° C.; 27° C.; 28° C.; 29° C.; 30° C.; 31° C.; 32° C.; 33° C.; 34° C.; 35° C.; 36° C.; 37° C.; 38° C.; 39° C.; 40° C.; 41° C.; 42° C.; 43° C.; 44° C.; 45° C.; 46° C.; 47° C.; 48° C.; 49° C.; or 50° C.

In some embodiments, the time at which the hydrogel incubates can be varied to vary the hydrogel stiffness and ECM fiber length. For example, the time at which the hydrogel incubates can be at least about 1 minute; at least about 2 minutes; at least about 3 minutes; at least about 4 minutes; at least about 5 minutes; at least about 6 minutes; at least about 7 minutes; at least about 8 minutes; at least about 9 minutes; at least about 10 minutes; at least about 11 minutes; at least about 12 minutes; at least about 13 minutes; at least about 14 minutes; at least about 15 minutes; at least about 16 minutes; at least about 17 minutes; at least about 18 minutes; at least about 19 minutes; at least about 20 minutes; at least about 21 minutes; at least about 22 minutes; at least about 23 minutes; at least about 24 minutes; at least about 25 minutes; at least about 26 minutes; at least about 27 minutes; at least about 28; at least about 29 minutes; at least about 30 minutes; at least about 31 minutes; at least about 32 minutes; at least about 33 minutes; at least about 34 minutes; at least about 35 minutes; at least about 36 minutes; at least about 37 minutes; at least about 38 minutes; at least about 39 minutes; at least about 40 minutes; at least about 41 minutes; at least about 42 minutes; at least about 43 minutes; at least about 44 minutes; at least about 45 minutes; at least about 46 minutes; at least about 47 minutes; at least about 48 minutes; at least about 49 minutes; at least about 50 minutes; at least about 51 minutes; at least about 52 minutes; at least about 53 minutes; at least about 54 minutes; at least about 55 minutes; at least about 56 minutes; at least about 57 minutes; at least about 58 minutes; at least about 59 minutes; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

Chemically modified PA gels with desired stiffness can be fabricated on any material, such as glass (e.g., coverslip), plastic, or a cell culture vessel. For example, PA precursor solutions can be prepared by mixing varying amounts of acrylamide, bis-acrylamide, and N-ethanal acrylamide.

### Patterned Proteins

The substrate or hydrogels as described herein can be coated or patterned with proteins. Without being limited to any particular theory, the proteins patterned on the substrate are selected and patterned in a manner representative of the in vivo microenvironment of podocytes. Non-limiting examples of proteins suitable for patterning on the substrate of the disclosed podocyte culture system include ECM proteins, glomerular receptors, glomerular ligands, antibodies to podocyte cell surface proteins, natural or artificial ligands/receptors for podocyte cell surface proteins and any other suitable protein without limitation. By way of non-limiting example, extracellular matrix (ECM) proteins are patterned on the substrate to provide a microenvironment to cultured podocytes that is representative of the in vivo microenvironment of glomeruli in a healthy condition or various disease states. By way of non-limiting example, one or more ECM proteins are patterned or coated onto the substrate.

Referring again to FIGS. 19 and 20, the ECM-patterned substrate 100 further includes at least one extracellular matrix (ECM) protein deposited on the exposed surface 106 of the substrate 102 in some aspects. In various aspects, the at least one ECM proteins are deposited on the exposed surface 106 of the substrate 102 in one or more patterns. In some aspects, the one or more ECM proteins are deposited in an ECM layer 110 over at least a portion of the exposed surface 106, as illustrated in FIG. 20. In other aspects, the one or more ECM proteins are deposited over the exposed surface 106 of the substrate 102 (see FIG. 19) or over the ECM layer 110 (see FIG. 20) as a plurality of patterned elements 104. In various aspects, each of the patterned elements 104 contains the same single ECM protein or combination of ECM proteins. In various other aspects, the patterned elements 104 contain a single ECM protein or combinations of ECM proteins that are different from at least a portion of other patterned elements 104.

In various aspects, the patterned elements may be deposited in any suitable element footprint, pattern, or arrangement without limitation in order to provide an in vitro microenvironment representative of the in vivo glomerular microenvironment of podocytes. Non-limiting examples of suitable element footprints include elongate footprints such as rectangular or elliptical, circular, polygonal footprints such as triangular, square, and the like, and any other suitable element footprint. Non-limiting examples of suitable patterns and arrangements of the patterned elements include uniform distributions, clumped distributions, random distributions, groupings of patterned elements with similar protein compositions, spatial gradients of protein contents of patterned elements along one or more gradient axes according to protein composition, and any other suitable pattern.

In some aspects, the protein content of the patterned elements are ECM proteins representative of proteins of the GBM in health and/or disease/injury, including, but not limited to, elastins, laminins such as Lam-521, collagens such as collagen IV, and fibronectins. Additional extracellular matrix (ECM) proteins suitable for patterning on the substrate of the disclosed podocyte culture system are described in Frantz et al. The extracellular matrix at a glance J Cell Sci. 2010 Dec. 15; 123(24): 4195-4200.

In other aspects, the ECM protein can be a collagen protein such as fibrillar (Type I, II, III, V, XI), non-fibrillar, FACIT (Fibril Associated Collagens with Interrupted Triple Helices) (Type IX, XII, XIV, XIX, XXI), short chain (Type VIII, X), basement membrane (Type IV), multiplexin (Multiple Triple Helix domains with Interruptions) (Type XV, XVIII), MACIT (Membrane Associated Collagens with Interrupted Triple Helices) (Type XIII, XVII), or other types including type VI and VII). The most common types of collagen include type I: skin, tendon, vasculature, organs, bone (main component of the organic part of bone); type II: cartilage (main collagenous component of cartilage); type III: reticulate (main component of reticular fibers), commonly found alongside type I; type IV: forms basal lamina, the epithelium-secreted layer of the basement membrane; and type V: cell surfaces, hair, and placenta.

In various aspects, the ECM-patterned substrates may be produced using any suitable method without limitation. In some aspects, the ECM proteins may be patterned onto the hydrogel substrate using standard lithographic techniques as described in detail in the Examples below. Briefly, a silicon master with the desired micropattern design is prepared and used to produce PDMS stamps. The PDMS stamps are contacted with ECM solutions and the ECM-coated stamps and the hydrogel substrate surface are dried before contacting the stamp with the hydrogel surface to transfer the ECM proteins in the desired micropattern design onto the substrate surface.

### Incubation of Podocytes and Additional Cell Types Using Podocyte Culture System

In various aspects, the podocyte culture system disclosed herein is used to culture podocytes in the presence of different patterns or combinations of ECM proteins, other glomerular cells, candidate active compounds, and any combination thereof. The culture system is configured to provide a microenvironment representative of an in vivo glomerular microenvironment. In some embodiments, the system can be fabricated for immediate use or in the form of a kit as described below. In some embodiments, the system can be frozen or chilled for later use.

In various aspects, the podocytes are obtained from mouse or human glomeruli. In some aspects, the glomeruli are isolated using a differential adhesion method and the podocytes are then isolated from the glomeruli. In other aspects, the podocytes are isolated from the glomeruli using magnetic beads. In some embodiments, a kidney sample containing glomeruli is collected as a biopsy from a patient. In some embodiments, podocytes are collected from a urine sample. Non-limiting examples of methods of obtaining podocytes are described in additional detail in the Examples below.

In some embodiments, the podocyte culture system is used to incubate a podocyte monoculture. In other embodiments, the podocyte culture system is used to incubate a co-culture of podocytes and one or more other cell types representative of the in vivo microenvironment of the podocytes in healthy or diseased conditions. Non-limiting examples of suitable other cell types include epithelial cells, endothelial cells, interstitial cells, mesangial cells, and immune cells.

### Kidney, Podocyte, or Glomerular Diseases, Disorders, or Conditions

In various aspects, the podocyte culture system is configured to provide a microenvironment representative of an in vivo microenvironment within healthy, diseased, and/or injured glomerular tissues.

Kidney, podocyte, or glomerular diseases, disorders, or conditions that can negatively impact glomerular health can be advanced diabetic glomerulosclerosis, acute kidney injury (AKI), acute post-streptococcal glomerulonephritis (PSGN), acute renal failure, Alport syndrome, bacterial endocarditis-caused glomerular disease, chronic kidney disease, congenital nephrotic syndrome (CNS), cyclosporine A treatment induced ER stress, diabetic nephropathy (DN), focal segmental glomerulosclerosis (FSGS), genetic mutations of renal proteins, glomerular disease, glomerular diseases associated with proteinuria, glomerulonephritis, glomerulosclerosis, Goodpasture's syndrome, hereditary nephritis, hereditary proteinuric disease, HIV-associated glomerular disease, infection-related glomerular disease, IgA nephropathy, inflammation induced renal injury, ischemia-reperfusion-induced acute kidney injury or proteinuria, kidney disease associated with missense mutations in nephrin or podocin, kidney disease associated with underglycosylation of nephrin, kidney hypoxia, medullary cystic kidney disease, membranous nephropathy, minimal change disease (MCD), nephrotic syndrome (NS), nodular glomerular sclerosis, osmolar contrast-induced renal injury, protein overload, puromycin aminonucleoside nephrosis (PAN), renal fibrosis, steroid-sensitive nephrotic syndrome, steroid-resistant nephrotic syndrome, systemic lupus erythematosus (SLE), total kidney failure, tunicamycin-induced acute kidney injury or proteinuria, or polycystic kidney disease. GA glomerular disease can be associated with mechanobiological dysregulation of the components that comprise the three-layered filtration barrier: the endothelial cells, the glomerular basement membrane (GBM), and especially the podocytes.

### Therapeutic Methods

In various aspects, candidate therapeutic agents may be screened using the podocyte culture system describe herein to evaluate efficacy. The candidate therapeutic agents may be screened for use in a process of treating, preventing, or reversing kidney, podocyte, or glomerular diseases, disorders, or conditions in a subject in need of administration of a therapeutically effective amount of a candidate therapeutic agent, so as to correct cellular homeostasis. In various aspects, the candidate therapeutic agents may be contacted with podocytes cultured using the disclosed podocyte culture system and the effects of the candidate therapeutic agents on the podocytes may be observed.

The therapeutic methods described herein are generally performed on a subject in need thereof. A subject in need of the therapeutic methods described herein can be a subject having, diagnosed with, suspected of having, or at risk for developing kidney, podocyte, or glomerular diseases, disorders, or conditions. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a candidate therapeutic agent is, for example, an amount that would cause the desired therapeutic effect in a subject while minimizing undesired side effects. In various embodiments, an effective amount of a candidate therapeutic agent described herein can substantially inhibit kidney, podocyte, or glomerular diseases, disorders, or conditions, slow the progress of kidney, podocyte, or glomerular diseases, disorders, or conditions, or limit the development of kidney, podocyte, or glomerular diseases, disorders, or conditions.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a candidate therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to correct cellular or podocyte homeostasis; substantially inhibit kidney, podocyte, or glomerular diseases, disorders, or conditions; slow the progress of kidney, podocyte, or glomerular diseases, disorders, or conditions; or limit the development of kidney, podocyte, or glomerular diseases, disorders, or conditions.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a candidate therapeutic agent can occur as a single event or over a time course of treatment. For example, a candidate therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for kidney, podocyte, or glomerular diseases, disorders, or conditions.

A candidate therapeutic agent can be administered simultaneously or sequentially with another agent, such as an antibiotic, an anti-inflammatory, or another agent. For example, a candidate therapeutic agent can be administered simultaneously with another agent, such as an antibiotic or an anti-inflammatory. Simultaneous administration can occur through administration of separate compositions, each containing one or more of a candidate therapeutic agent, an antibiotic, an anti-inflammatory, or another agent. Simultaneous administration can occur through administration of one composition containing two or more of a candidate therapeutic agent, an antibiotic, an anti-inflammatory, or another agent. A candidate therapeutic agent can be administered sequentially with an antibiotic, an anti-inflammatory, or another agent. For example, a candidate therapeutic agent can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the candidate therapeutic agent may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a candidate therapeutic agent may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

In various aspects, the disclosed podocyte culture system may be used to screen potential cells generated for use in a cell therapy treatment of a kidney, podocyte, or glomerular disease, disorder, or condition. Candidate cells for cell therapy may be cocultured with podocytes using the podocyte culture system as described herein and evaluated to assess the effects of the candidate cell therapy on the podocytes.

Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation have gained significant interest from researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig-derived cells can be transplanted into humans. Xenogeneic cell therapies can involve human cell transplantation into experimental animal models for assessment of efficacy and safety or enable xenogeneic strategies for humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods for identifying therapeutics (e.g., small molecules) or therapeutic targets.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to patterned hydrogel substrates, components thereof, 96 well plates, podocyte cells, and any combination thereof. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

In some embodiments, the kit can include podocytes that are frozen and configured to be thawed and grown on the patterned hydrogel or other desired substrate. In some embodiments, the podocytes are packaged in a vial or a blister pack with elastic substrate deposited over the interior of the vial or pack. In some aspects, the container is lined with an ECM-patterned hydrogel layer to provide the podocytes with a microenvironment representative of in vivo glomerular conditions during transport and storage.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: A Novel Method for Drug Screening Using Mouse and Human Primary Podocytes Grown on Micropattern-Printed Hydrogels

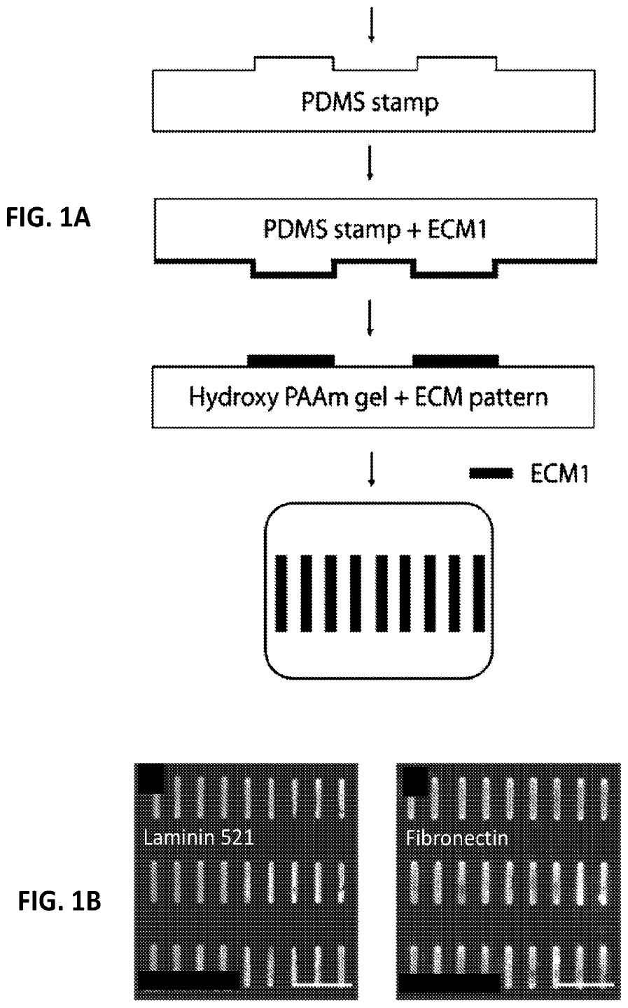

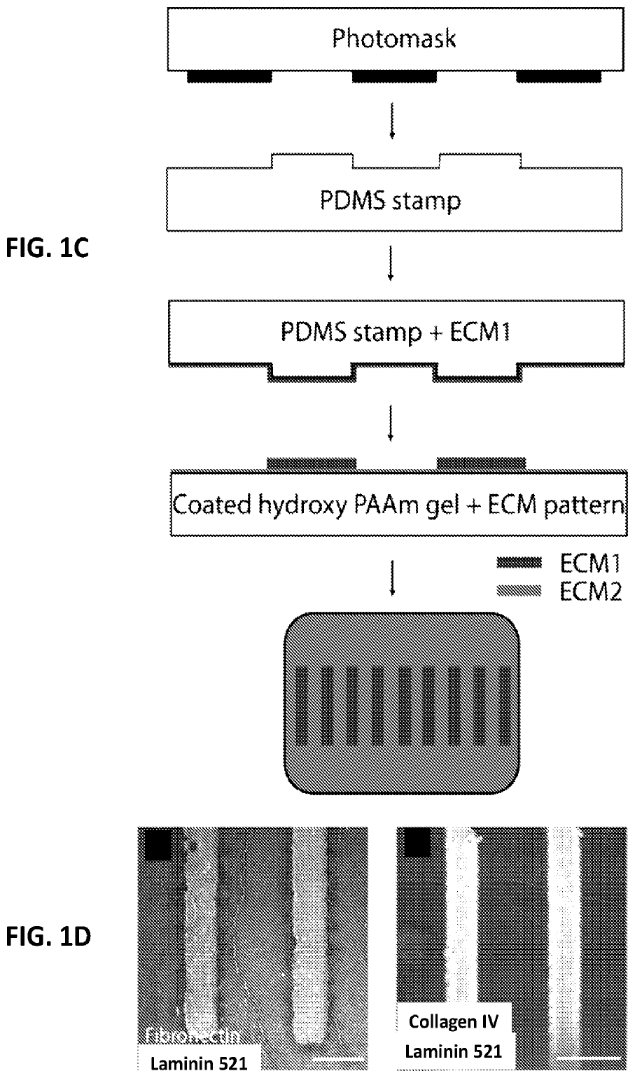

We developed a method for patient-specific drug screening for kidney disease. At the core of the new method are a device and a method to grow mouse and human primary kidney podocytes on a physiological substrate that is an improvement over what has been done before. The cells, which can be collected from a patient's urine, are grown on a hydrogel that, for the first time, has a physiologically relevant microenvironment for the cells and thereby enables observation of how the cells would respond in the body. This includes providing cells a substrate of the correct stiffness with extracellular matrix printed on it as micropatterns (see FIG. 1). This method allows us to identify how substrate stiffness affects the ability of a patient's podocytes to spread, to test the ECM requirement for their healthy signaling and/or homeostasis, and to determine the patient-specific effect of drugs on correcting this homeostasis. A key hallmark of the success of this system is that primary podocytes grown on it upregulate sarcomere-like structures, a pattern that has only ever before been observed in injured podocytes in vivo. This is hypothesized to be an attempt by the podocytes to adapt to their new environment as they try to recover from pathophysiological conditions.

Our system and the pattern observed could be used to screen for small molecules that change podocyte behavior, which can suggest a way to treat glomerular diseases.

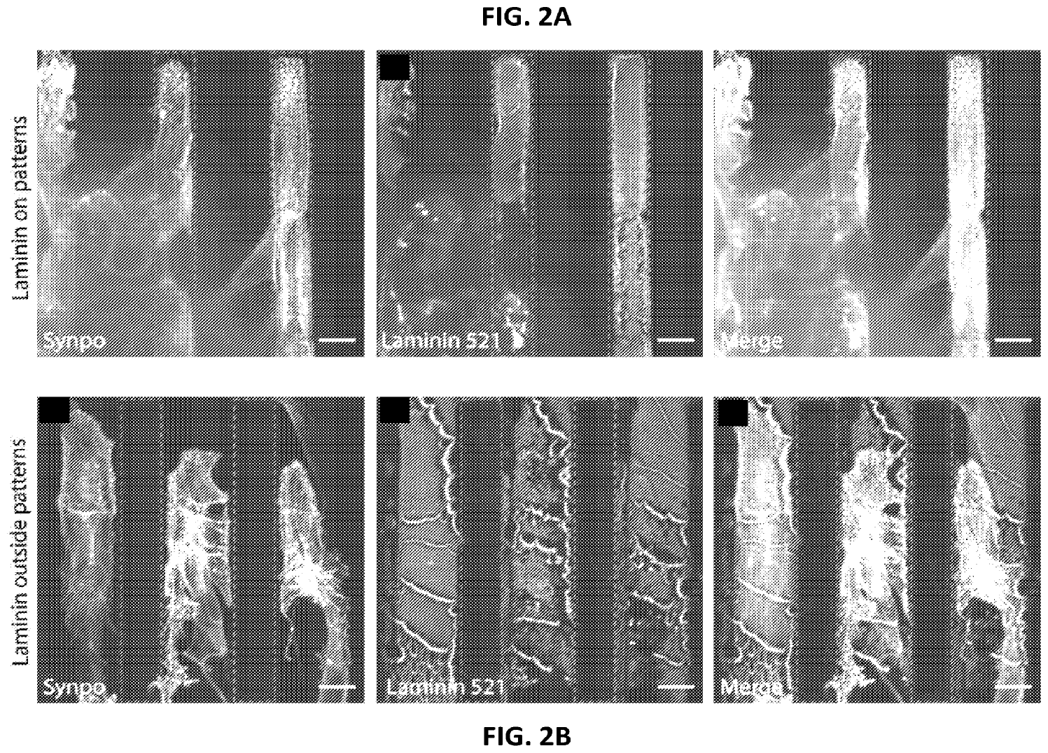

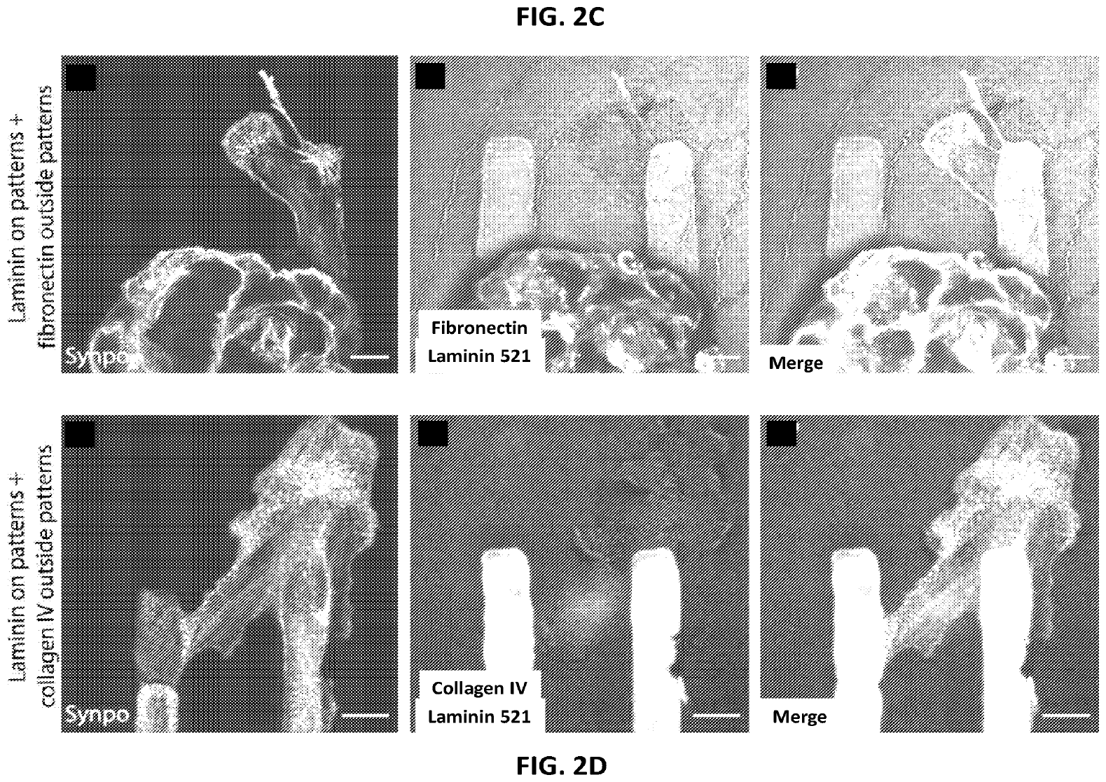

We adapted the method and redesigned the approach to allow for drug screening using 96-well plates (see FIG. 2). The approach will speed the process of hydrogel assembly as well as the ECM microprinting on each well in 96-well plates. Such an approach will allow us to seed primary podocytes (mouse or human) and observe the changes in the sarcomere-like structures when different small molecules are added to the wells. We will use a robotic system to add the drugs and image the samples afterward.

The source of the primary podocytes could be mouse or human donors. Human podocytes can be isolated as follows:

1) Podocytes can be isolated from urine: every day healthy individuals and kidney disease patients shed ˜3000 podocytes into the urine.

2) Podocytes can be grown out of iPSC-derived kidney organoids. These organoids contain glomeruli that can be isolated and cultured on the micropattern-printed hydrogel.

Two fundamental commercial problems are solved. First, it is difficult to grow human podocytes in culture while maintaining their unique physiologic characteristics. Growing human podocytes on a hydrogel with the correct stiffness and extracellular matrix substrate is an improvement over what has been used before. This approach allowed us to observe novel behaviors of the podocytes on laminin substrates, which we hypothesize are changes that reflect healthy adaptive mechanisms. Second, no physiologically accurate culture system currently exists to enable patient-specific drug screening. We adapted our initial approach to fit 96 well-plates that can support drug screens for active small molecules that can change the cellular and cytoskeletal patterns in order to identify novel drug targets.

### Example 2: Mechanosensitive, Synaptopodin-Templating, Sarcomere-Like Structures Revealed in a Culture Model of Kidney Podocyte Injury

This example describes a novel in vitro system that enables the study of podocyte injury outside of the native podocyte microenvironment.

### Abstract

Chronic kidney diseases are widespread and incurable. The biophysical mechanisms underlying them are unclear, in part because material systems for reconstituting the microenvironment of the relevant kidney cells are limited. A critical question is how kidney podocytes (glomerular epithelial cells) regenerate the foot processes of the filtration apparatus following injury. Recently identified sarcomere-like structures (SLSs) with periodically spaced myosin IIA (a contractile protein) and synaptopodin (an actin-associated protein) appear in injured podocytes in vivo. It was hypothesized that SLSs template synaptopodin in the initial stages of recovery, this hypothesis was tested by developing a culture system that models both kidney physiology and pathophysiology. SLSs were observed in vitro for the first time as podocytes migrated out of harvested kidney glomeruli onto micropatterns of physiologically relevant proteins. SLSs emerged over two days, and cells formed foot process-like extensions from these periodically spaced proteins. SLS distributions and morphology were sensitive to actomyosin inhibitors, substrate stiffness, and extracellular matrix proteins associated with pathology. These results indicate a role for mechanobiological factors in podocyte recovery from injury and suggest SLSs as a target for therapeutic intervention.

### Introduction

Kidney glomerular diseases result in damage to the kidney's filtration apparatus and often cause chronic kidney disease and kidney failure with no cure. One barrier to devising successful treatments for these diseases is the lack of a full understanding of the biophysical mechanisms that underlie them. Glomerular disease may be associated with mechanobiological dysregulation of the components that comprise the three-layered filtration barrier: the endothelial cells, the glomerular basement membrane (GBM), and especially the podocytes. Podocytes are unique epithelial cells with hundreds of foot processes that interdigitate with those of adjacent podocytes; these are connected by slit diaphragms, unique intercellular junctions that are critical for filtration. However, the inability to study podocytes outside of their native microenvironment has left key gaps in our knowledge of how they maintain the intricate structures that enable filtration and of how injury and healing progress. To address this critical need, a culture system that enables studying podocyte injury outside of their native microenvironment was developed.

Previous work on podocyte mechanobiology includes a wealth of studies in vitro using immortalized mouse and human podocyte cell lines and, more recently, using primary mouse podocytes. However, this work has occurred with cells cultured on glass substrates. The physiological stiffness range of the podocyte microenvironment ranges from the ˜0.74-kPa modulus of decellularized glomeruli to the ˜2.4-kPa modulus of the GBM, and stiffness increases with pathologies such as diabetes. Kidney podocyte cell lines adopt a more physiological phenotype when cultured on slightly stiffer polyacrylamide (PAAm) hydrogels, in the 0.9- to 9.9-kPa range. Glass is a million-fold stiffer. Such a mismatch is known to affect cell structure and spreading and a broad range of mechanobiological responses. To solve these problems, a range of biomimetic platforms has thus been proposed, including culturing immortalized podocytes on soft hydrogel-based or polydimethylsiloxane (PDMS) substrates, curved substrates, or micropatterned glass substrates or in three-dimensional (3D) collagen gels. However, these systems do not produce the structures and cell shapes observed in vivo, partially because they fail to sufficiently reconstitute the microenvironment that podocytes require. Because of the fact that the mechano-responsiveness of podocytes in health and disease is thus largely unknown, a biomimetic platform that combines primary podocytes, physiologically relevant substrate stiffnesses, physiologic or pathophysiologic extracellular matrix (ECM) proteins, and physiologically relevant cell confinement is developed.

The gap in knowledge that is addressed relates to the mechanisms that podocytes use to repair connections to the GBM and their adjacent podocyte neighbors. Studies of glomeruli in vivo have revealed that in mouse models of kidney diseases, podocytes contain the normally absent contractile protein myosin IIA in the basal aspect of the areas of foot process effacement, and the injured podocytes develop sarcomere-like structures (SLSs). On the basis of these in vivo data, it is speculated that SLSs are associated with responses to mechanobiological cues associated with pathology and possibly associated with podocyte migration and healing. However, because SLSs are difficult to study in vivo because of their nanoscale size and because they have not been observed in any current in vitro systems, this hypothesis has not been possible to test. Therefore, the usefulness of our culture system is demonstrated by testing this hypothesis and identifying mechano-biological factors that collectively regulate SLSs.

### Results

Micropatterned Substrates with Defined Protein Patterns Representative of Healthy and Pathologic GBM.

Using a freshly fabricated PDMS mold, patterns of ECM proteins representative of the GBM in health and injury were printed (FIG. 1A). To represent physiologic GBM, a PAAm hydrogel of defined stiffness was used to microprint human laminin α5β2γ1 trimer (Lam-521), the dominant laminin trimer in the GBM. To represent injury, Lam-521 was replaced with fibronectin, a GBM protein that is synthesized in certain glomerular diseases and podocyte injuries (25). Using immunofluorescence staining of the hydrogel with antibodies specific to the different ECM proteins, patterns were visualized as 105 μm by 7 μm microprints (FIG. 1B). To microprint two different ECM substrates, the hydrogel was coated with the first ECM protein and left to dry before microprinting the second ECM protein atop the first (FIG. 1C). In this way, Lam-521 micropatterns were printed atop PAAm hydrogels that were coated with either collagen IV, representative of a healthy GBM, or fibronectin, representative of abnormal GBM (FIG. 1D). Note that the lines of protein condensation associated with drying of the surface proteins did not appear to affect the mechanics of the cellular microenvironment, as evident from strain fields associated with cellular contraction.

Podocytes from Isolated Glomeruli Migrate onto Micropatterned Lam-521

To test which micropatterned ECM proteins best support podocytes, mouse glomeruli were isolated and cultured on hydrogels coated with Lam-521, collagen IV, or fibronectin. Culturing the glomeruli for 2 days followed by immunostaining using antibodies against synaptopodin, a podocyte-specific cytoskeletal marker, and human laminin α5, to identify the location of the Lam-521 micropatterns (FIG. 2), revealed that the podocytes populated micropatterns coated with Lam-521 (FIG. 2A) but not those coated with fibronectin. Furthermore, the PAAm hydrogel was coated with Lam-521 and micropatterned protein-free patches onto the substrate using a PDMS stamp that removed Lam-521, leaving an inverse of the previous Lam-521 micropatterns interdigitated with microscale patches of exposed PAAm. Using this setup to culture glomeruli showed that podocytes migrated onto the Lam-521-coated areas but not the bare micropatterned PAAm hydrogel areas (FIG. 2B, areas outside of the red boxes).

Next, PAAm hydrogels coated with fibronectin and micropatterned with Lam-521 were used. As expected, glomeruli attached only to the Lam-521 micropatterns, and podocytes only migrated onto them, whereas other cell types readily spread on both substrates. Podocytes could extend protrusions into regions covered with fibronectin (FIG. 2C) but only after they migrated onto the Lam-521 micropatterns. Sometimes, they migrated across regions of fibronectin to neighboring Lam-521 micropatterns. Similar results were observed for collagen IV-coated PAAm hydrogels with Lam-521 micropatterns, with most of the podocytes confined to the Lam-521 micropatterned regions; podocytes occasionally extended processes onto collagen IV from their Lam-521 foundations (FIG. 2D).

Primary Podocytes Present with a Mat of SLSs on Micropatterned Substrates

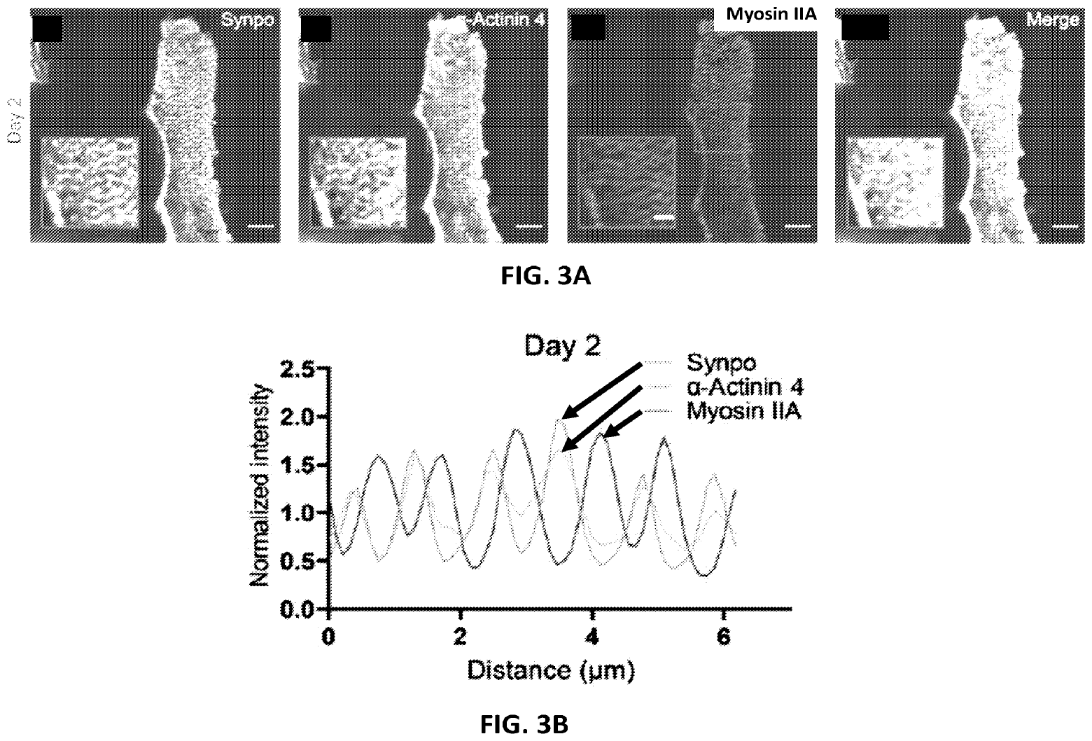

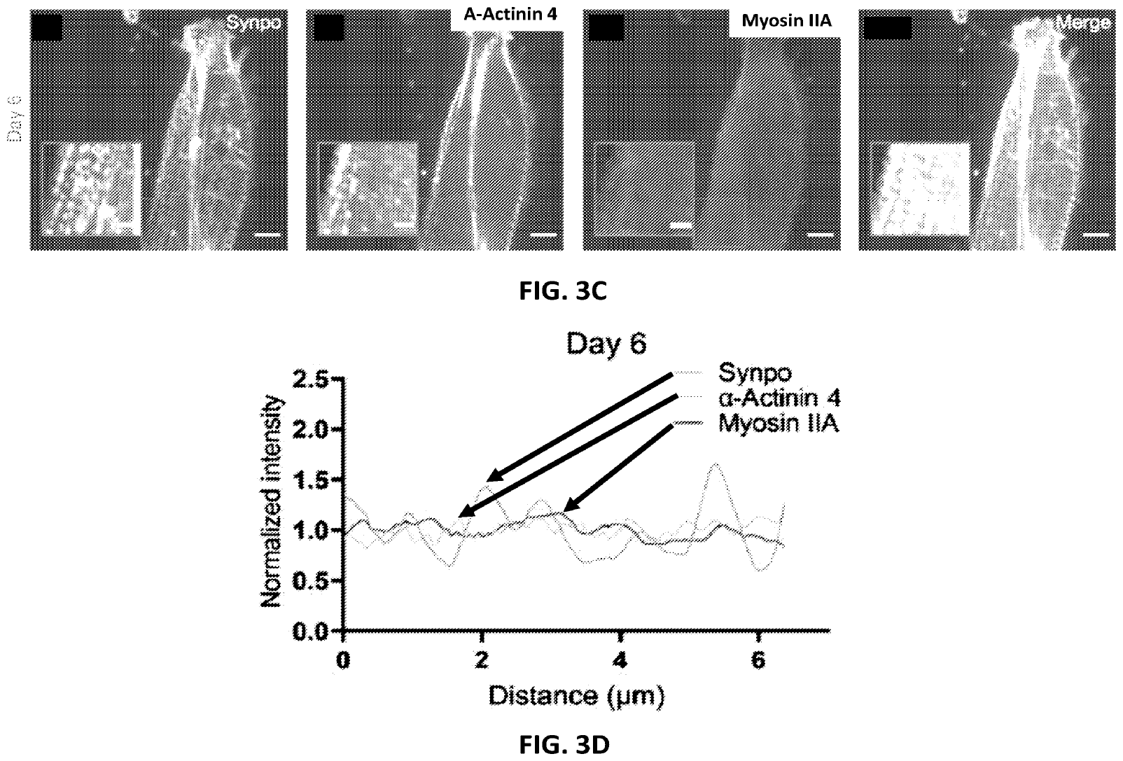

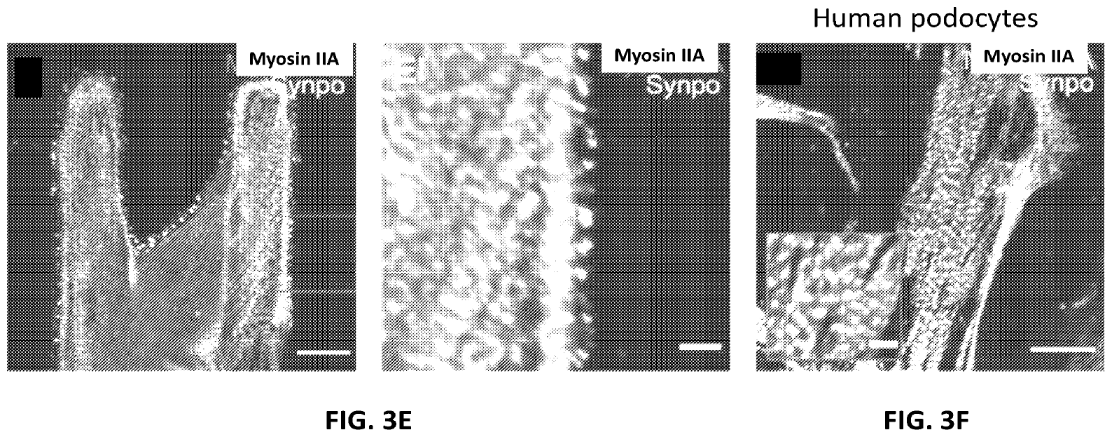

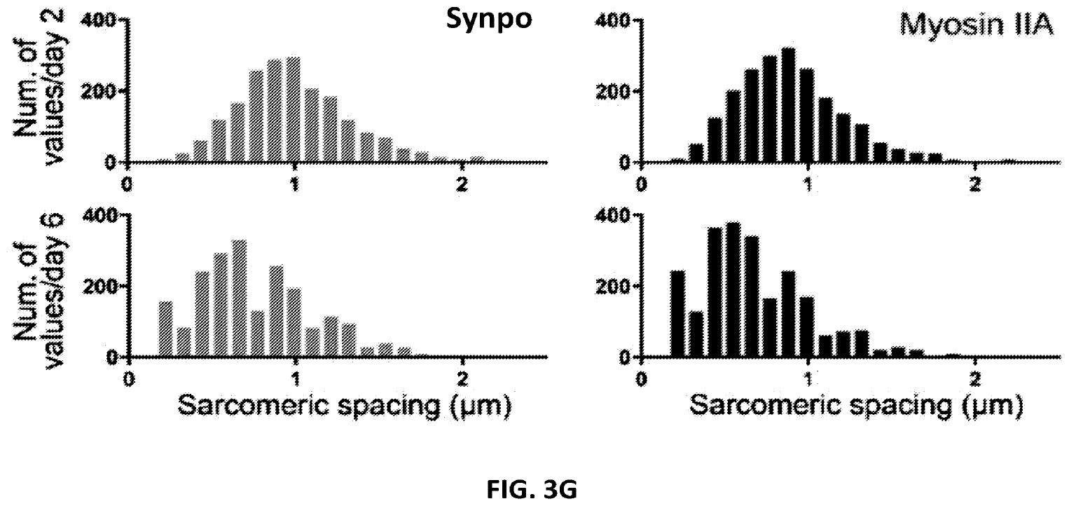

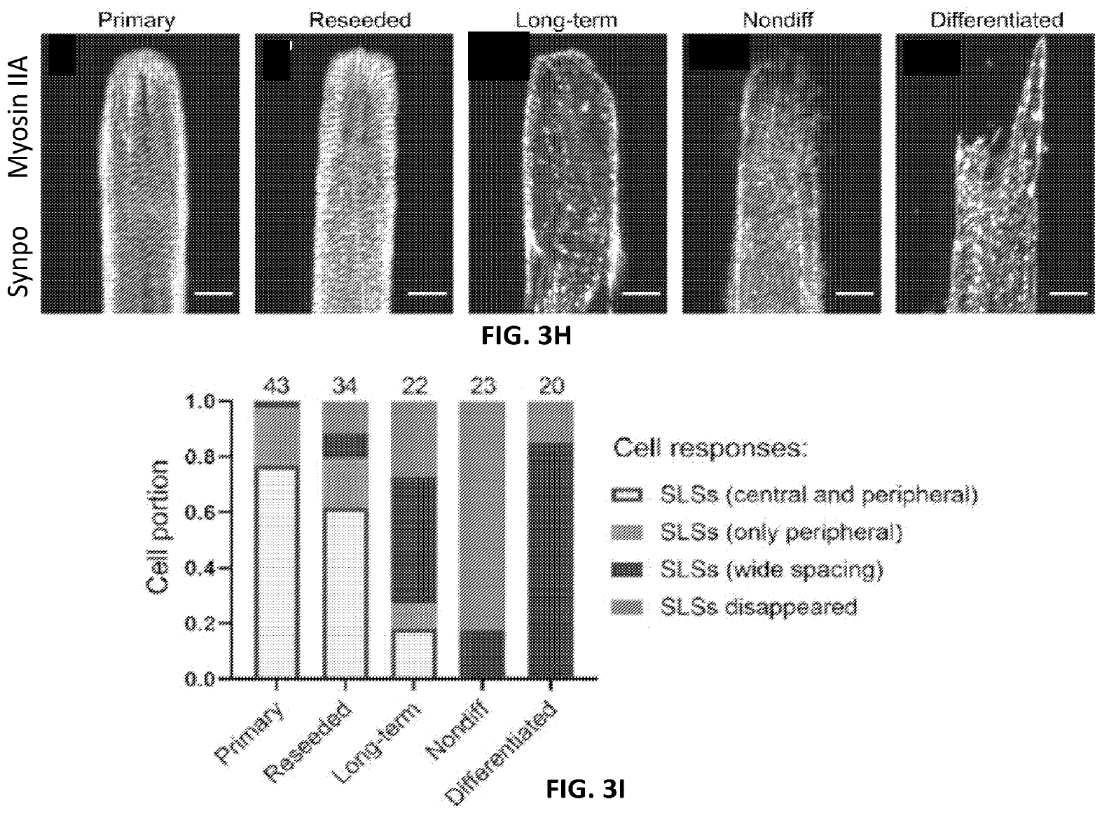

SLSs have been described in injured podocytes in vivo but have never been observed in cultured podocytes. Immunostaining of primary podocytes migrating out of the glomeruli onto the micropatterned hydrogels showed high numbers of SLSs 2 days after seeding of glomeruli onto the Lam-521 micropatterns (FIG. 3). These contained two markers of contractile activity: (i) the motor protein myosin IIA and (ii) synaptopodin, an actin-binding protein present in podocytes, arranged in a sarcomeric pattern of alternating synaptopodin and myosin IIA (FIG. 3A). α-Actinin 4, the major α-actinin isoform present in podocytes, was present in a striated pattern in registry with synaptopodin (FIG. 3A). This periodicity was confirmed by quantifying fluorescence intensity along an axis paralleling the Lam-521 micropatterns (FIG. 3B). It was next asked whether these spread podocytes still have slit diaphragm proteins. An antibody against nephrin showed a lack of nephrin at the cell membrane, perhaps due to the fact that the cells are confined to the micropattern but require cell-cell contact to up-regulate slit diaphragm protein expression.

To investigate whether the primary podocytes that up-regulate SLSs acquire muscle-related characteristics, the cultured podocytes were immunostained for α-smooth muscle actin (α-SMA). No α-SMA was detected in podocytes, although other glomerular cell types were positive for α-SMA. This lack of α-SMA suggests that spreading primary podocytes presenting SLSs do not acquire muscle-related characteristics.

SLSs are Transient and can Form without Synaptopodin

It was next asked whether SLSs represent a transient or a permanent structure in podocytes. To address this, podocytes were followed over a prolonged culture interval of 6 days. When compared to the 2-day cultures, the SLSs in 6-day cultures showed a less pronounced synaptopodin pattern and a nearly dissolved myosin IIA or α-actinin 4 pattern (FIG. 3B), as evident from the spatial intensity map (FIG. 3D) and from the histogram of sarcomeric spacing (FIG. 3G) or cell sorting (FIG. 3I). This suggests that the SLSs became attenuated over time.

Next, the role of synaptopodin in the formation of SLSs was evaluated. Using glomeruli isolated from Synpo−/− mice to culture podocytes that were then stained with α-actinin 4 and myosin IIA antibodies, SLSs in the spreading podocytes with a striated pattern similar to that of wild type could be observed. This indicates that synaptopodin is not required for the formation of SLSs.

SLSs are Associated with Newly Formed Peripheral Foot Process-Like Protrusions

To assess the function of the SLSs, their role in establishing connectivity to other cells was evaluated. Using two ECM proteins, Lam-521 on the micropatterns and fibronectin outside the micropatterned area, it was observed that podocytes with SLSs sent out protrusions extending over areas coated with fibronectin (FIG. 2C). Some processes extended to connect podocytes across fibronectin patterns that were several cell widths apart. Moreover, the periodic synaptopodin patterning in the SLSs was associated with the spacing between the newly formed synaptopodin-positive protrusions, speculated to be immature foot process-like structures (FIG. 3E).

To confirm that SLSs are relevant to human podocytes, the protocols were applied to freshly harvested glomeruli taken from human kidney nephrectomy samples that were seeded onto the Lam-521 micropatterned hydrogels. Similar to the mouse glomeruli, human glomeruli attached to the Lam-521 micropatterns, although they took longer to attach when compared with mouse glomeruli. After 3 days of culture, sarcomeric structures could be observed in podocytes migrating from human glomeruli (FIG. 3F), confirming that SLSs are present in primary human podocytes when migrating onto the micropatterns.

### SLSs are Specific to Primary Podocytes

Similar to the primary podocytes at day 2, reseeded primary podocytes (see Materials and Methods) maintained synaptopodin- and myosin IIA-positive SLSs (FIGS. 3H and I). This experiment suggests that the formation of SLSs is a stable characteristic of primary podocytes regardless of the culture conditions. Next, whether the immortalized mouse podocyte (IMP) cell line presents with SLSs was evaluated. While undifferentiated IMPs showed no clear SLS patterns, differentiated IMPs [using VRAD (vitamin D3, retinoic acid, and dexamethasone-supplemented DMEM/F12) differentiation media for 7 days] presented with loose synaptopodin+/myosin+ actin cables (FIGS. 3H and I). In contrast to the SLSs in primary podocytes, the distances between the periodic synaptopodin patterns in the differentiated IMPs were notably larger, and myosin no longer showed the same striated pattern.

### SLSs are Mechanosensitive

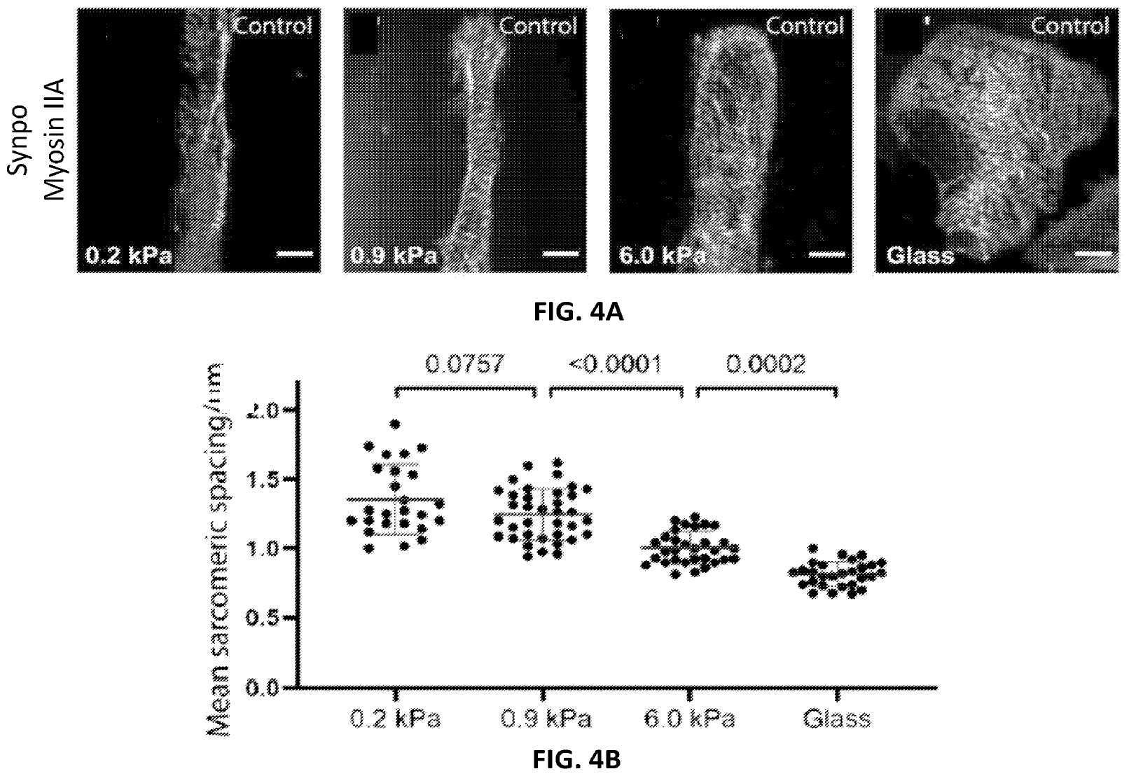

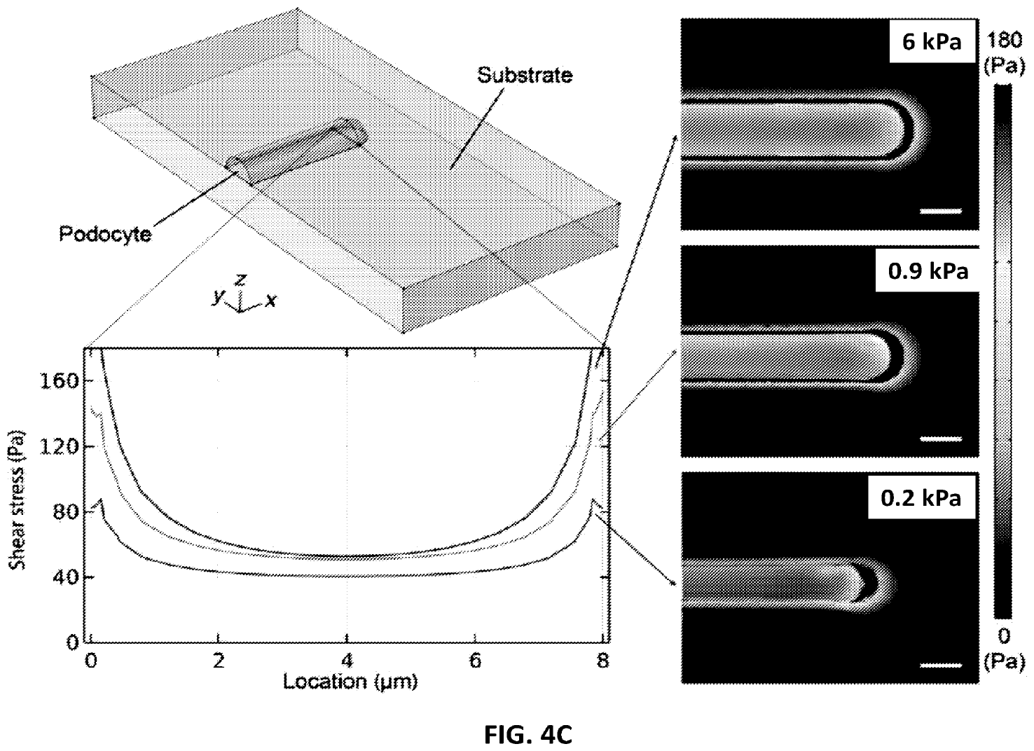

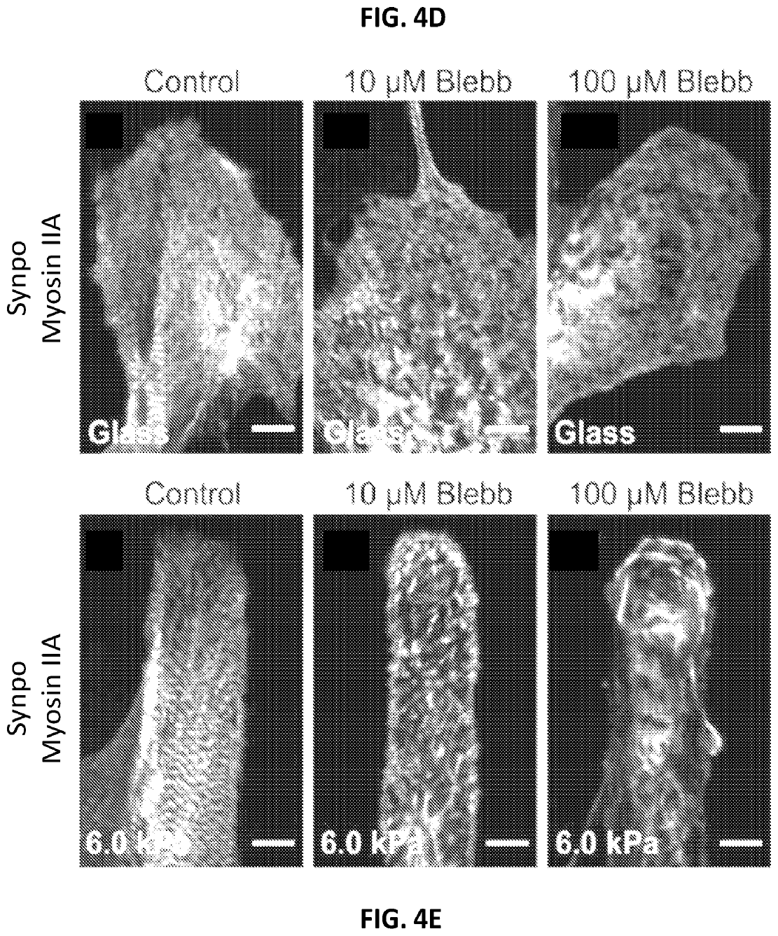

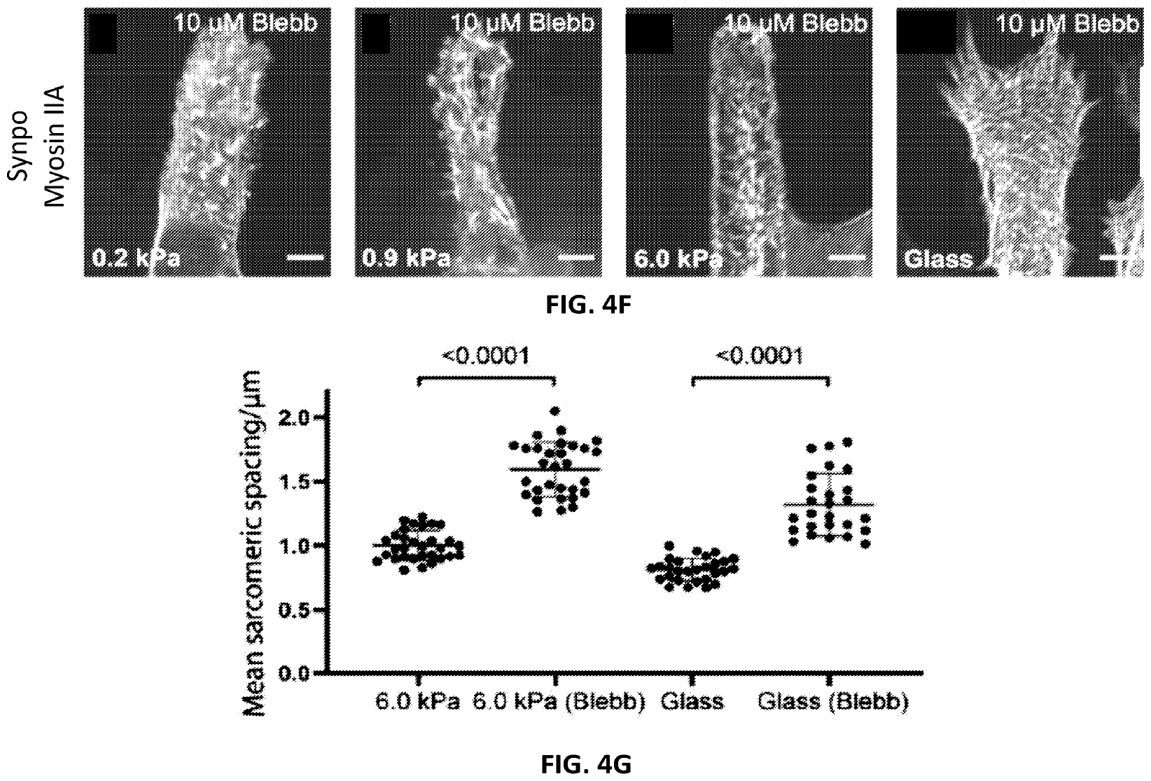

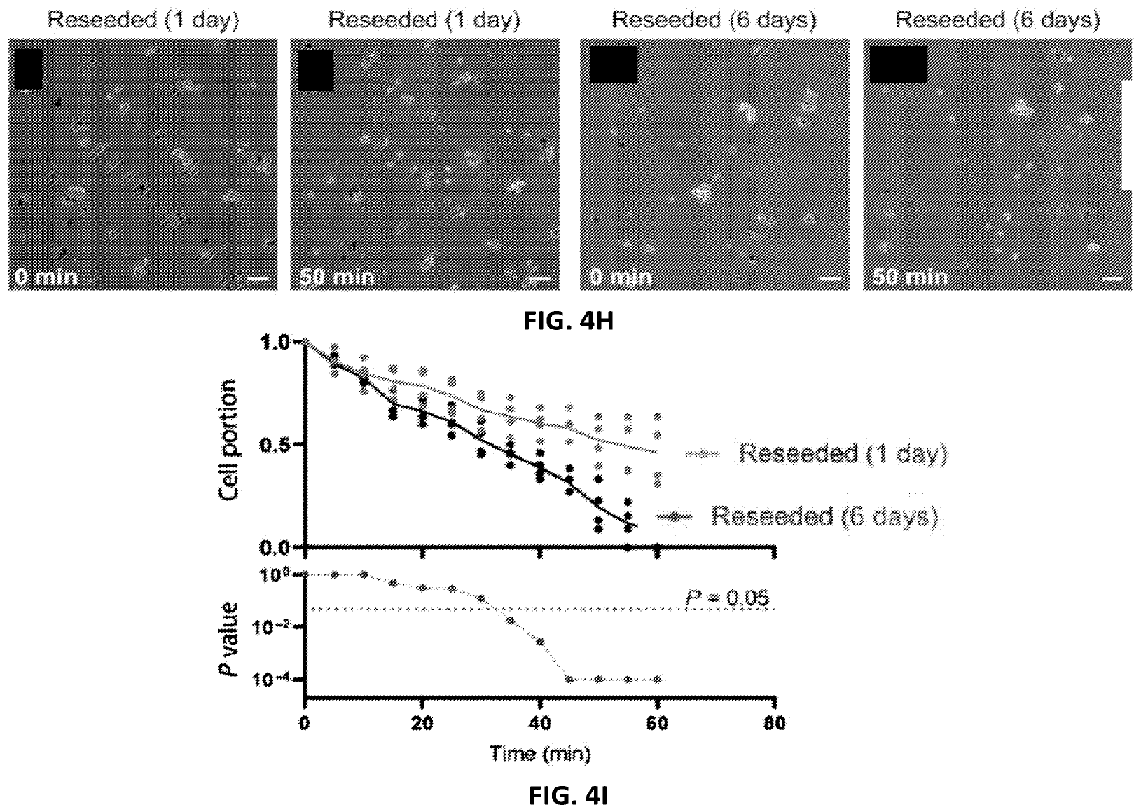

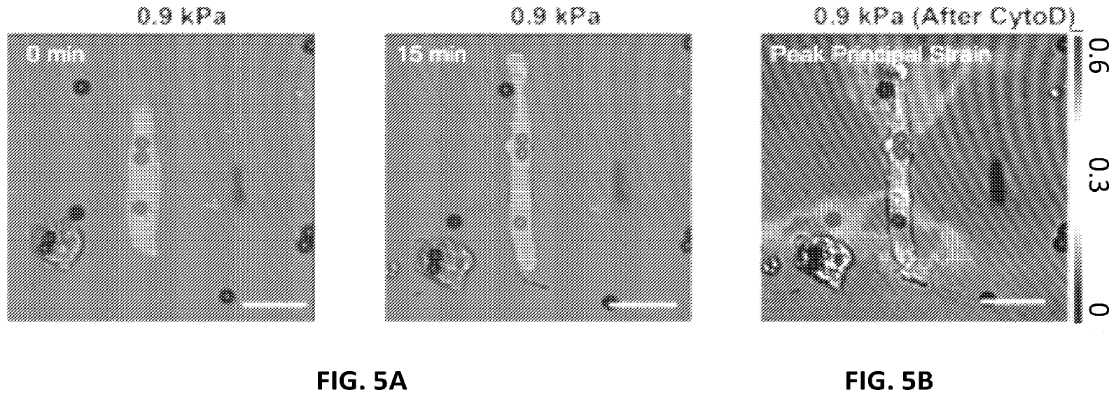

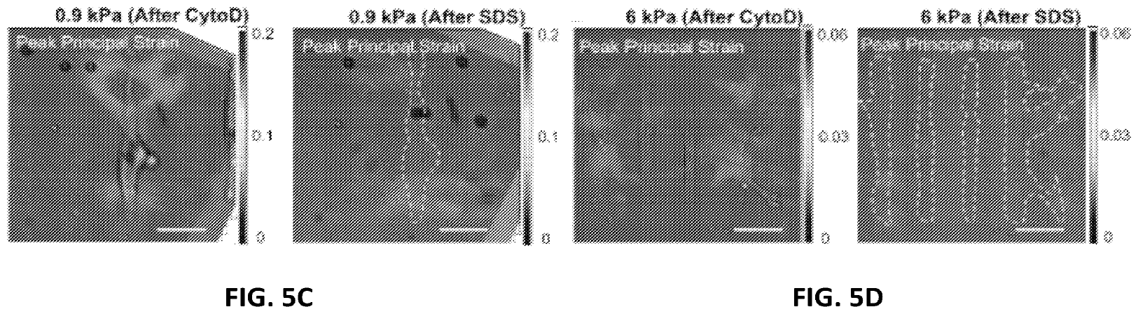

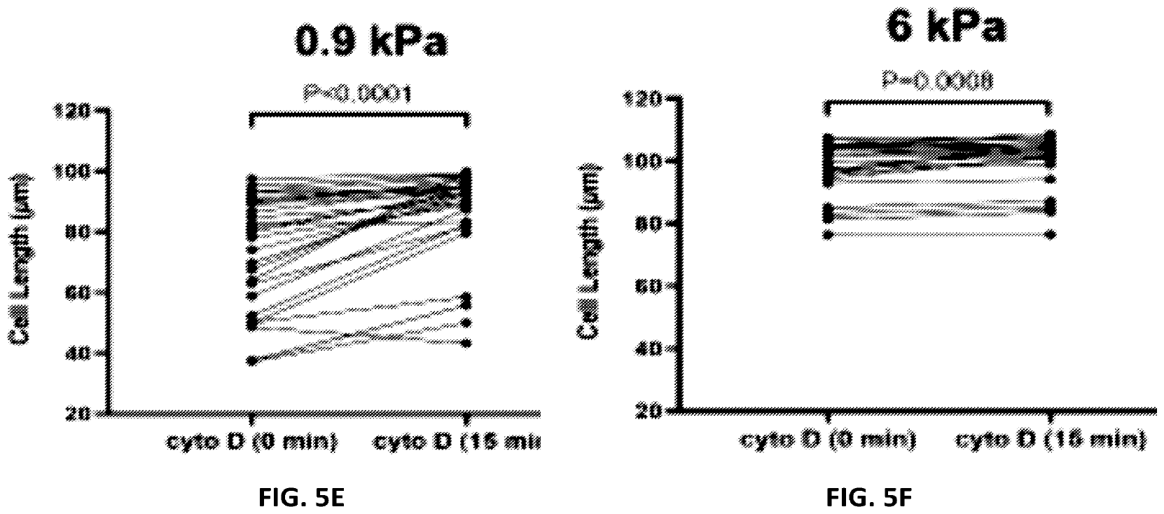

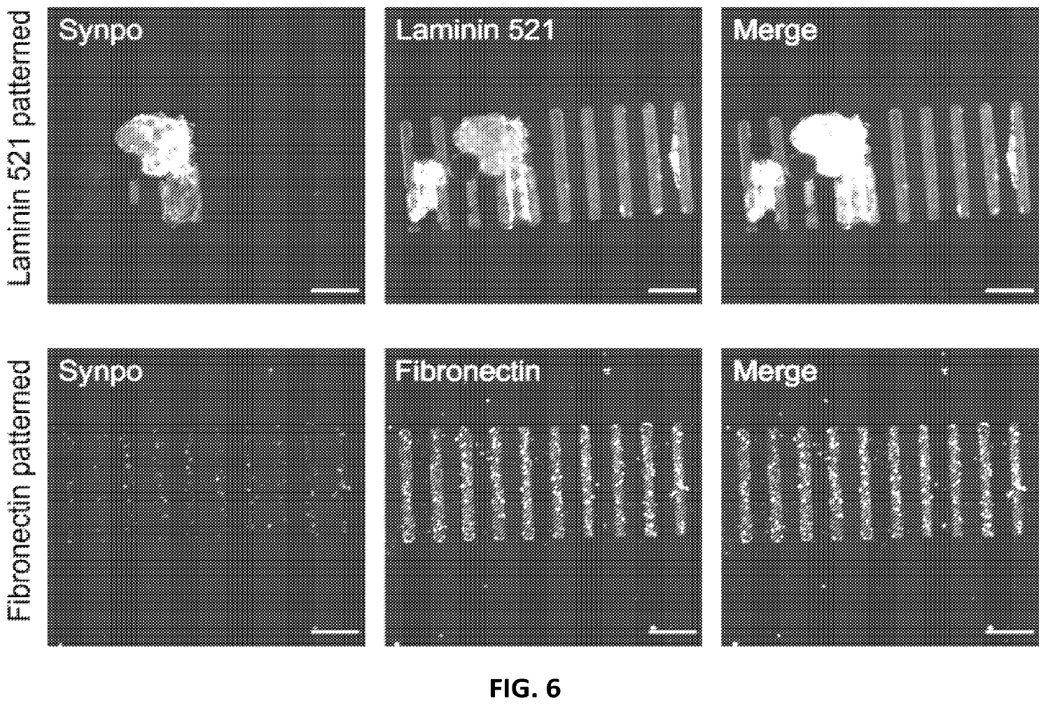

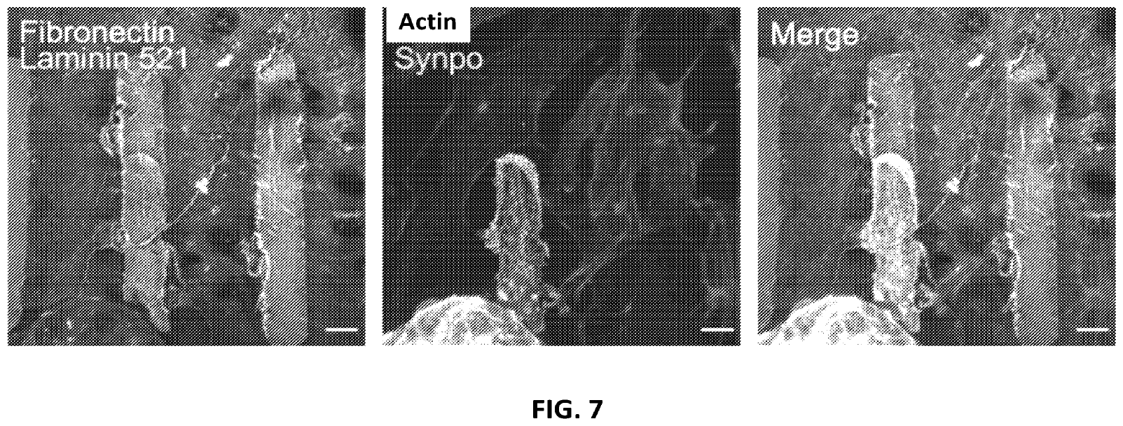

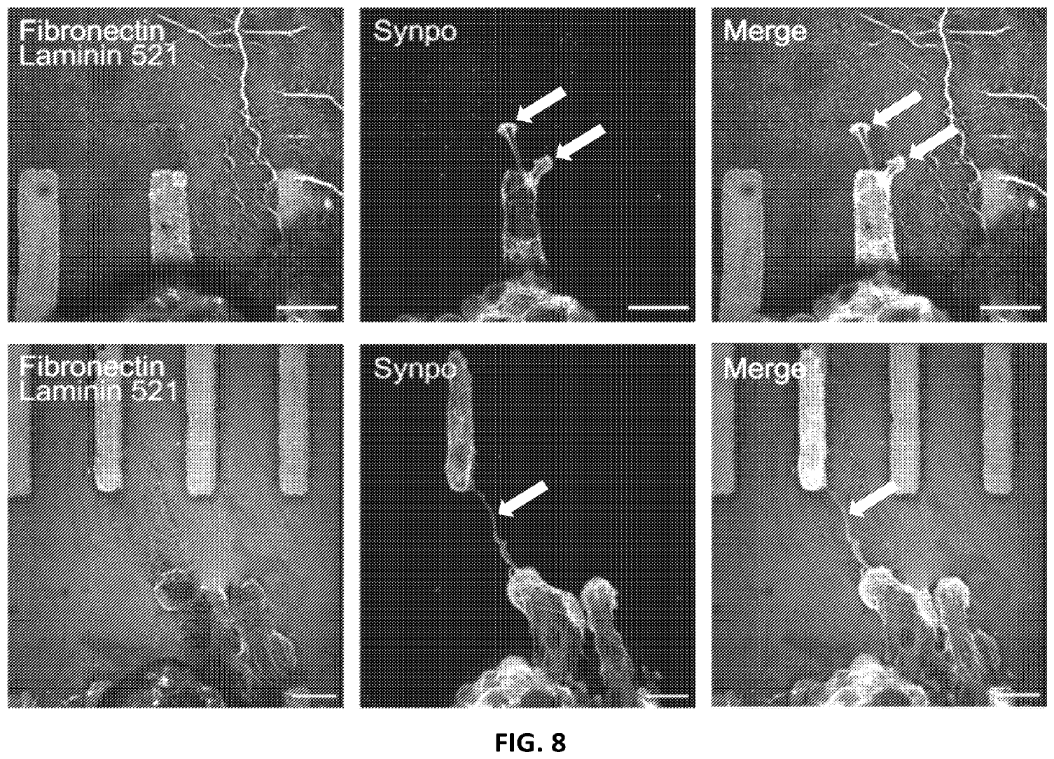

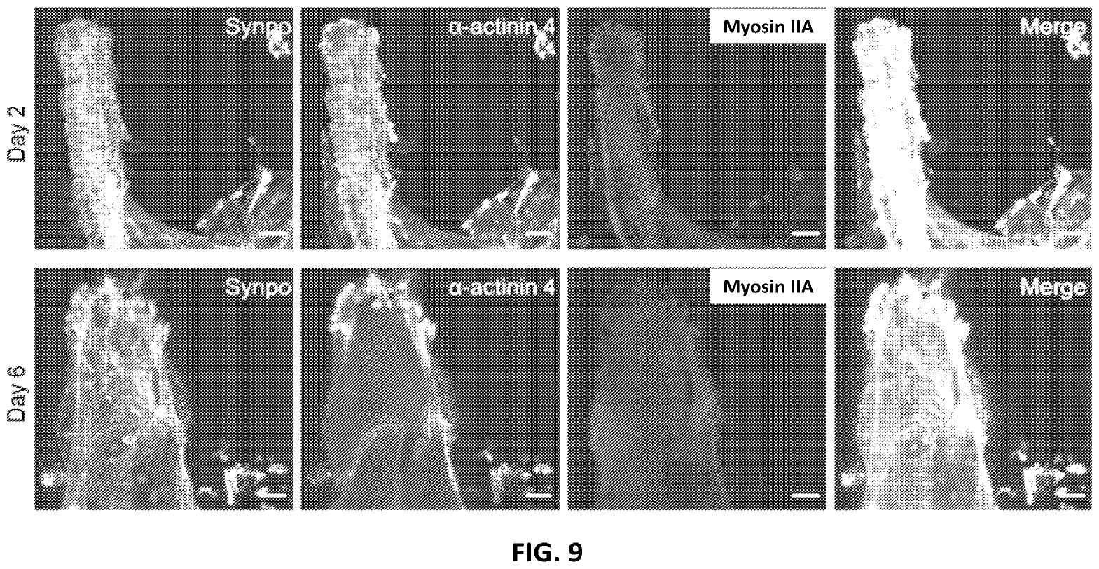

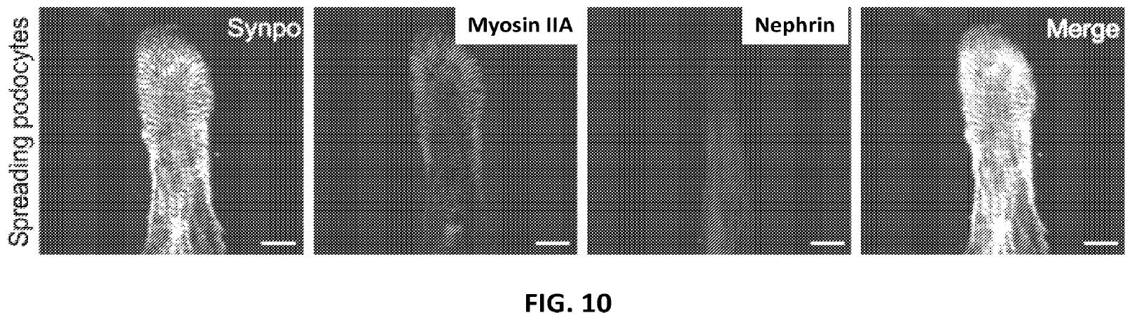

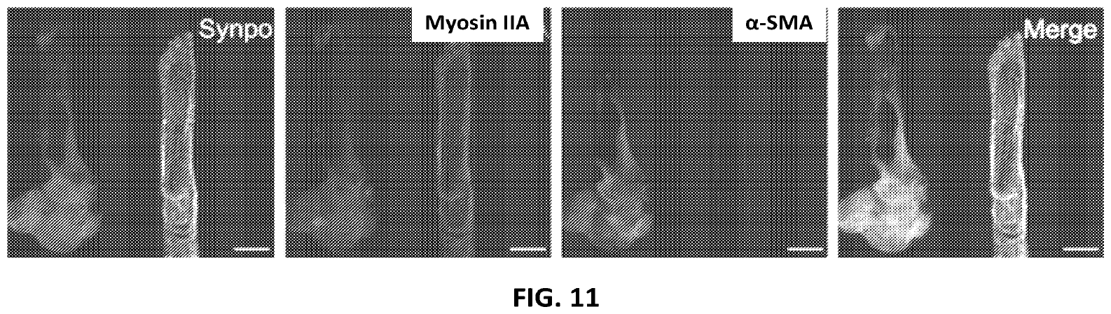

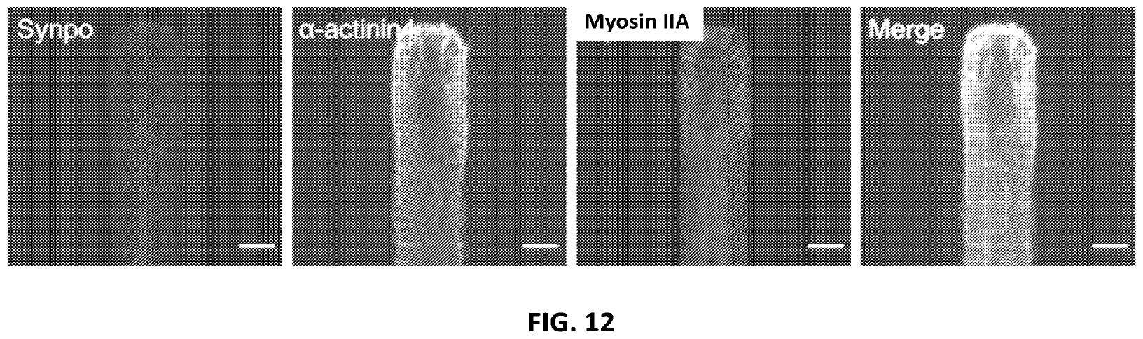

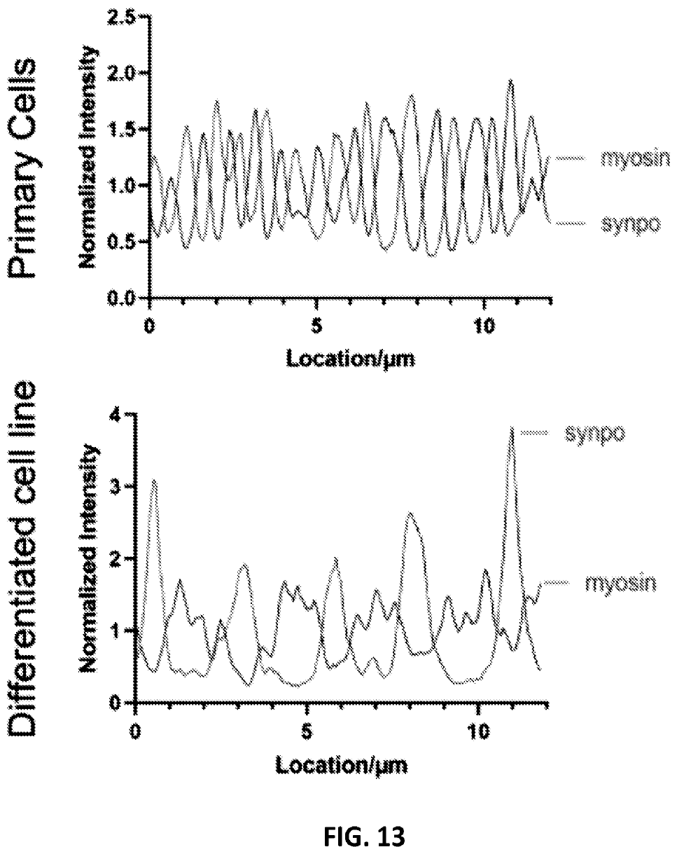

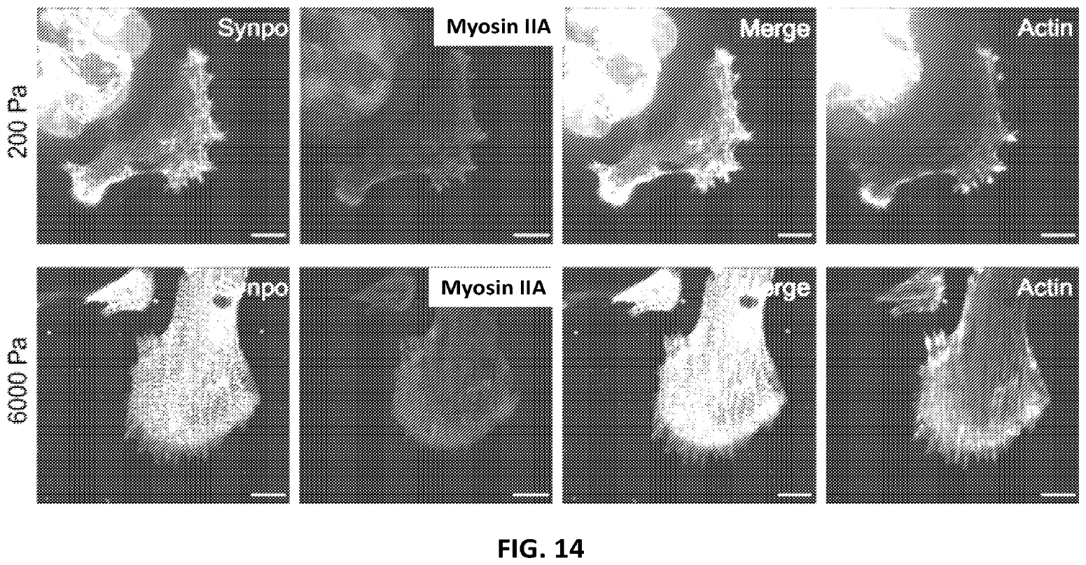

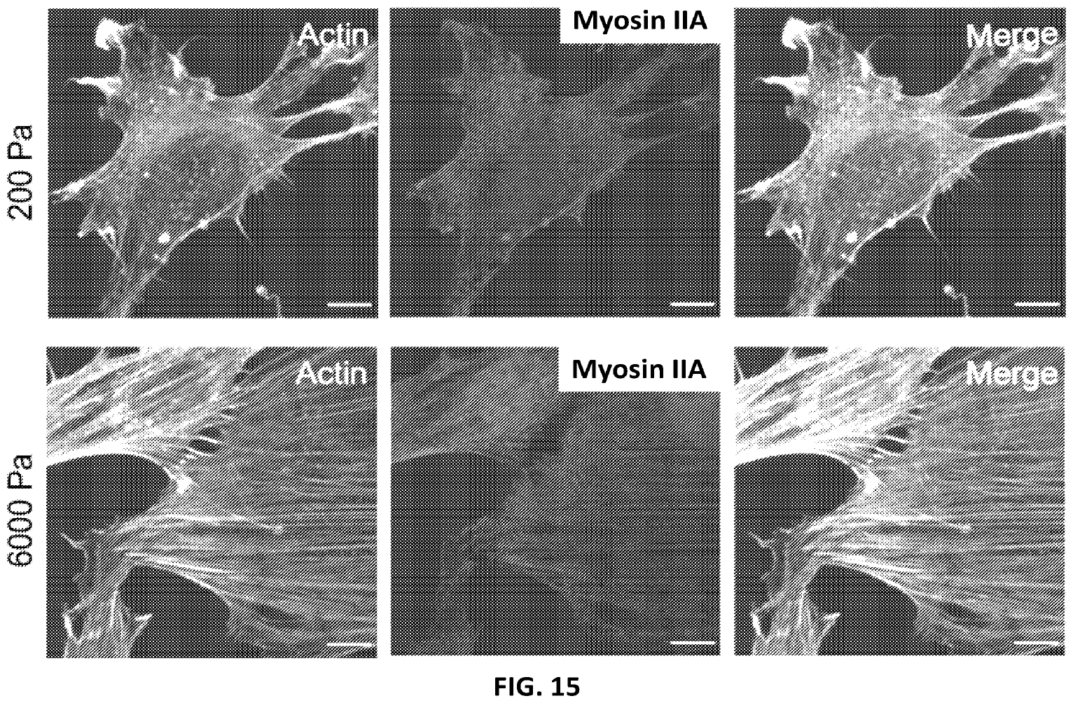

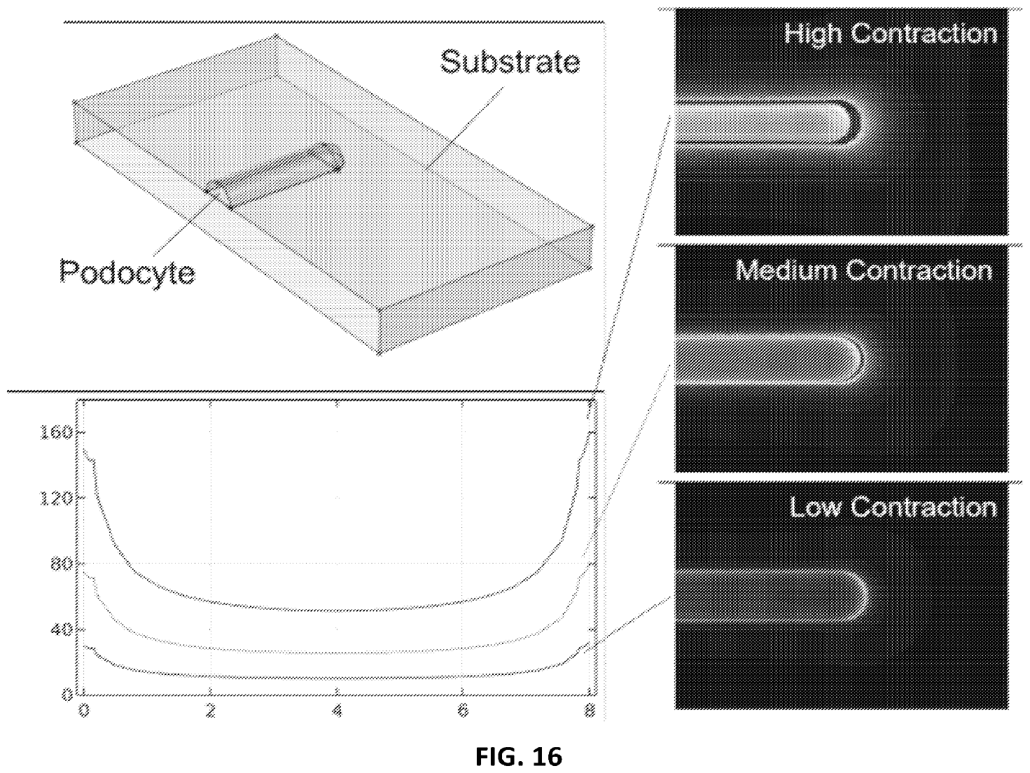

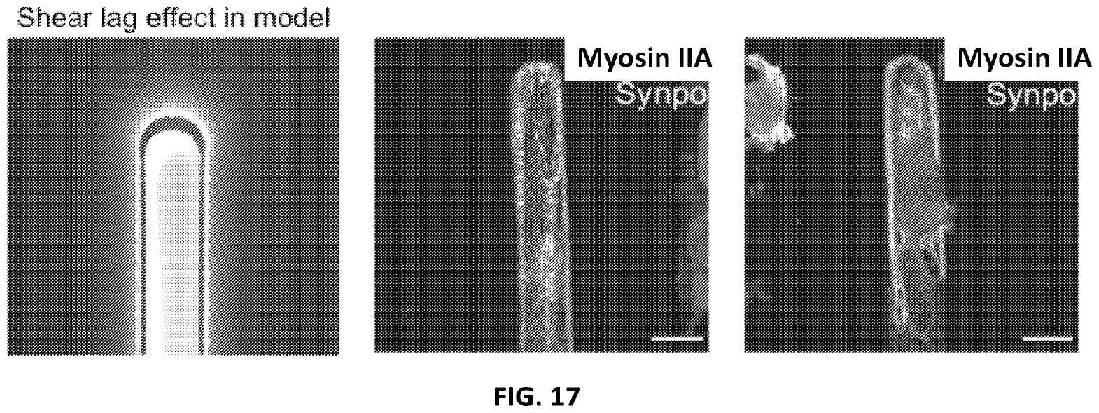

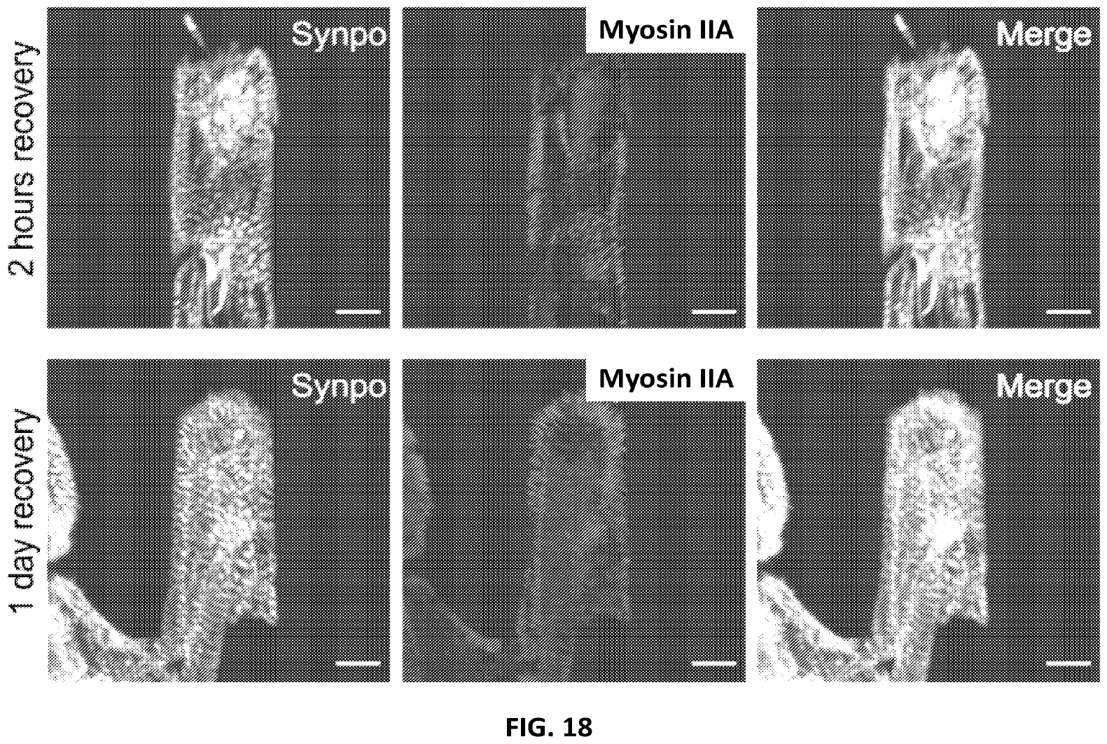

It was next asked whether SLSs are sensitive to their mechanical microenvironment. PAAm hydrogels with elastic moduli in the physiologic range (0.2 and 0.9 kPa) and pathophysiologic ranges (6.0 kPa, representative of GBM stiffening under hypertension or glycation, and cover glass, as a fully rigid substratum) were micropatterned or coated with Lam-521. After a 2-day culture of glomeruli, SLSs were evident in podocytes on the substrata of all moduli, even without the micropatterns, but the spacing of the sarcomeric patterning was wider on the softer hydrogels than on the stiffer hydrogels and cover glass (FIG. 4A). The periodic synaptopodin distribution from 120 cells over three replicate experiments showed substantial differences between softer hydrogels (0.2 or 0.9 kPa) and stiffer hydrogels or glass (FIG. 4B). As an additional control, 3T3 mouse fibroblasts were also cultured on hydrogels of two different stiffnesses. Similar to podocytes, 3T3 cells presented with more stress fibers on the stiff hydrogel (i.e., 6.0 kPa) than on the soft hydrogel (i.e., 0.2 kPa).

### SLSs Require Actomyosin Contractility

Given that mechanosensitivity and mechanosensing in other cell types arise from actomyosin contractility, it was next asked whether SLSs can be controlled by modulating myosin II using the drug blebbistatin. In cells such as smooth muscle cells and fibroblasts, reductions in substrate stiffness reduce the resistance to acto-myosin contractility and can thereby destabilize stress fibers and focal adhesions that require force for stability.

Numerical simulations revealed that stresses were highest along the periphery of cells and at their distal ends (FIG. 4C). Stresses also increased with substrate modulus (FIG. 4C) and were attenuated by reduced cell contractility. Following a standard model of mechanosensitive stabilization and growth of stress fibers, these simulations thus predicted that stress fibers disassemble in the interior of cells and at the proximal ends when actomyosin contractility is attenuated either by actomyosin inhibition or by reducing substrate modulus. Simulations also predicted that cell width decreases with decreasing substrate modulus (FIG. 4C).

To test these predictions, two concentrations of blebbistatin were first applied to podocytes cultured on substrates with high stiffness (6.0-kPa PAAm hydrogels and cover glass), micropatterned or coated with Lam-521. Myosin II inhibition via blebbistatin disrupted the sarcomeric structures of SLSs in a way that depended on the dose, substrate stiffness, and position (FIGS. 4D and E), consistent with the model predictions. On both substrates, 10 μM blebbistatin caused aggregation of synaptopodin in the center of the cells and increased sarcomeric spacing in SLSs that persisted at the cell periphery (FIGS. 4D and E), as predicted by the model. Higher doses (100 μM) of blebbistatin nearly eliminated SLSs (FIGS. 4D and E). Further experiments applied a milder myosin inhibition using 2 μM blebbistatin to verify this shear lag effect. This was insufficient to disrupt the SLSs at the cell periphery but sufficient to disrupt those in the centers of the cells, and the corresponding distribution of SLSs in podocytes followed the model's predictions.

A second prediction of the model was that soft substrates lead to lower cell contractility and thus enhance the effects of actomyosin inhibition. To test this, whether a minimum stiffness threshold was required for the formation of SLSs in cells treated with 10 nM blebbistatin was explored. In podocytes treated with 10 nM blebbistatin and cultured on 6.0-kPa hydrogels or glass substrates, SLSs formed but with wider sarcomeric spacing (FIGS. 4F and G). When this treatment was applied to podocytes cultured on 0.2- or 0.9-kPa hydrogels, representative of understressed GBMs under hypotensive conditions due to the nonlinearity of GBM, SLSs failed to form, with no sarcomeric structures evident (FIG. 4F). This supported the hypothesis that actomyosin-based mechanosensation governs the formation of SLSs and that a threshold of developed tension is required.

Last, the model predicted that cells would contract laterally on substrates of lower stiffness: The reduced contractile stresses associated with low substrate stiffness would be outweighed by the decreased resistance to lateral contraction. This prediction was borne out by our experiments, with cells showing narrower bodies on substrates of lower modulus (FIG. 4A).

### SLS Formation can be Rescued Through Removal of Actomyosin Inhibition

To test the hypothesis that mechanobiological factors alone can drive SLS formation, it was asked whether SLSs susceptible to actomyosin inhibition are reversible after washing out blebbistatin. After 2 hours of inhibition, cells were left to recover for another 2 hours or 1 day and then stained for synaptopodin and myosin IIA. SLSs appeared as early as 2 hours following the removal of blebbistatin, although some condensates of synaptopodin were still evident. After 1 day of recovery, these condensates disappeared, and SLSs returned.

### Contractile SLSs Promote Podocyte Adhesion to the Underlying Substrate

A potential role for SLSs in vivo is to enable podocytes to maintain their adhesion to the GBM even when faced with complex and changing mechanical forces within the glomerular microenvironment. The contractile nature of SLSs is likely paramount for this role. To directly study podocyte contractility, reseeded primary podocytes were used, as more than 70% contain SLSs (FIG. 3I). Video recording of podocytes after disrupting their actin network with cytochalasin D (CytoD) showed significant cell relaxation after adding the drug. This suggests that the cells were under contractile strain before actin depolymerization. The movements of fluorescently labeled microbeads embedded in the hydrogel with either 0.9- or 6-kPa stiffnesses allowed us to calculate the strength of cell contractility. As shown by the spatial variation of the first principal Green-Lagrange strain in the adhesion plane, most cells relaxed and extended as the contraction was attenuated by the action of the inhibitor CytoD on both stiffnesses. Additional strain calculation associated with the removal of cells using 10% SDS after the CytoD treatment suggests that very little strain energy is stored within the cell after CytoD treatment.

Last, to determine the adhesive strength of primary podocytes, cell detachment assays were performed using Cellstripper. Compared with freshly plated podocytes (i.e., 1 day after reseeding, when more than 70% have SLSs), podocytes cultured for a longer term (i.e., 6 days after reseeding, when fewer SLSs) showed higher tendencies for faster detachment (FIGS. 4H and I), suggesting an important role for SLSs in stabilizing podocyte adhesion.

### DISCUSSION

The micropatterned culture system enabled the visualization of SLSs, which had previously been observed only in injured podocytes in vivo. The results demonstrated the presence of SLSs in primary podocytes that had migrated from both mouse and human glomeruli and established SLSs as mechanosensitive and dynamic structures.

The results further suggest that SLSs are associated with a healing phenotype, with SLSs linked to conditions associated with podocyte healing or antidetachment responses. Bands rich in myosin IIA were found to alternate in SLSs with bands rich in both synaptopodin and α-actinin 4. A role in healing is further suggested, because dominant mutations of α-actinin 4 are associated with diminished podocyte injury resistance, including focal segmental glomerulosclerosis. One possibility is that SLSs template periodic synaptopodin for podocyte healing and recovery, as suggested by the synaptopodin-rich processes that were observed; these are reminiscent of the foot processes that are required for normal podocyte-podocyte connectivity and podocyte function. These protrusions resembled foot processes observed during development in native podocytes, further suggesting that they are representative of a healing phenotype. Because the periodic synaptopodin patterning in the SLSs was associated with the spacing between the newly formed synaptopodin-positive protrusions, the SLSs, once laid out, might act as a template for guiding foot process formation (FIG. 3E). Although no specific pathogenic phenotype has been observed in synaptopodin knockout mice at baseline, the loss of synaptopodin exacerbates both drug-induced and genetic kidney injuries. The podocyte culture system may be useful for further clarifying synaptopodin's roles in podocyte injury and kidney glomerular disease.

The observation that SLS band spacing decreased with increasing substrate stiffness suggests that they are, similar to SLSs in other cells, mechanosensitive. This is consistent with observations of the mechanosensitive formation of stress fibers observed in immortalized podocytes on soft gelatin substrates. The formation and organization of SLSs are also interesting: To achieve the ideal spacing in the SLSs for the templating of synaptopodin, there may be different mechanisms compared to the organization of sarcomeres in the myofibrils of muscle cells, especially considering the fact that sarcomeric spacing is usually between 1.5 and 3.5 μm in myofibrils, whereas it was determined the sarcomeric spacing in SLSs to be between 0.8 and 1 μm in spreading podocytes during culturing on hydrogels with physiologic stiffness. Note that this is comparable to striated α-actinin patterns along the stress fibers of other spreading epithelial cells. The spacing between the periodic striations of sarcomeric-like stress fibers varies depending on the cell types, with ˜0.5 μm in osteosarcoma cells and ˜1.6 μm in fibroblasts. The mechanisms that determine the different spacing and their relationship to the contractile forces are unknown.

In addition to templating, SLSs may serve as a buffer against mechanical perturbations that could cause podocyte detachment from the GBM and their loss into the urine. Perturbations include changes to extrinsic shear stresses associated with filtration that arise from pressure differences between the capillaries and Bowman's space (such as in the hypertension condition), as well as changes to intrinsic stresses as may occur in response to pathological stiffening of the GBM. The sarcomeric patterns of SLSs are reminiscent of sarcomeric contractile units in muscle and contractile nonmuscle cells, including contractile actin cables that comprise the ventral stress fibers and the transverse arcs. Similar to stress fibers in fibroblasts, SLSs in primary podocytes aligned in the direction of maximum stress, at a density that correlated with the amplitude of stress, and may serve to stabilize motor clutch bonds to the ECM. SLS alignment with the direction of cell spreading might power cell spreading and colonization of the GBM surrounding the glomerular capillaries.

The micropatterns used in this study were rectangles of approximately 20 μm in length, with length:width ratios of 14:1 to 8:1. This size range allowed the spreading of podocytes on the 2D hydrogel surfaces, while part of the podocytes remained in contact with the glomerulus. Thus, podocytes remained connected to their native 3D structure while extending into confined patterns that mimicked the constraints of the GBM. One limitation of the system is that the micropatterns limit the ability of podocytes to form foot processes and slit diaphragms, and future work should address this inadequacy. Extensions of this technology via nanopatterning may better replicate the cell microenvironment and perhaps spur the formation of foot processes. The GBM has a curvature that, although large in radius compared to the size of a podocyte foot process, may nevertheless be a factor in podocyte mechanobiology. Incorporating curvature with microprinting via surface engraving technologies may enable the exploration of these factors. Densely cultured rat primary podocytes were shown to form interdigitating cell processes upon the addition of heparin and all-trans retinoic acid to the media. Such an approach in combination with the culture system could allow the study of the relationship between the interdigitating foot process-like protrusions and SLSs. Last, fluid shear stresses are an additional factor that is likely important in podocyte homeostasis, and the addition of microfluidics to the system may constitute an important step forward. However, even with these limitations, the culture system has served to identify and validate essential biophysical mechanisms underlying SLSs, including mechanobiological factors that serve as potential therapeutic targets. SLSs may be a transient feature of podocyte healing or an advantageous response to injury and thus a target for intervention in kidney injury and disease.

### Materials and Methods

### Activation of Cover Glasses

To enable firm attachment of PAAm hydrogels to cover glasses, the cover glasses were washed twice in NaOH (0.1 M) for 5 min, rinsed with ddH2O, and dried before applying a thin layer of 3-(trimethoxysilyl) propyl acrylate for 1 hour at room temperature. They were then washed again in ddH2O and dried under nitrogen flow.

Preparation of Hydroxy-PAAm Hydrogels with Defined Elastic Modulus

Preparation of hydroxy-PAAm hydrogels with defined elastic modulus was achieved by cross-linking precursors on activated cover glasses via modification of established protocols. Hydroxy-PAAm hydrogels were prepared using acrylamide [3.2 to 6.4% (w/w) in Hepes (pH 7.4)], bis-acrylamide [0.03 to 0.16% (w/w) in Hepes (pH 7.4)], and N-hydroxyethyl acrylamide (HEA) [1.3% (w/w) in Hepes (pH 7.4)] together with ammonium persulfate (APS) and N-tetramethylenediamine (TEMED). After incubating the precross-linking solution (i.e., the acrylamide, the bis-acrylamide, and the HEA) for 30 min under vacuum to remove oxygen and thus prevent oxidation, the cross-linking agents were added and incubated for another 30 min at room temperature. For 5 ml of precross-linking solution, 2.5 μl of TEMED and 25 μl of 10% APS were added. To shape the hydrogel as a thin layer and protect it from oxidation, a clean, nonactivated cover glass was placed on top. After the hydrogel solidified, the sample was washed three times with ddH2O and left at 4° C. To control the stiffness of the hydrogel, both acrylamide and bis-acrylamide final concentrations were altered, with acrylamide varied from 3.2 to 6.4% (w/w), and bis-acrylamide varied from 0.03 to 0.16% (w/w). The moduli of hydrogels were measured using atomic force microscopy and were controlled to range in stiffness from 0.9 to 6.0 kPa, a range that encompasses that reported for GBM.

### Micropattern Stamps for Microprinting

A silicon master with the desired micropattern design was prepared using standard lithographic techniques. A PDMS stamp was prepared by mixing PDMS and a cross-linking agent at a 10:1 ratio. PDMS was degassed under vacuum to remove air bubbles. The molding mixture was then added to a container in the presence of the silicon master template, and set inside an oven (VWR) at 60° C. for 2 hours to initiate the cross-linking.

Next, the PDMS stamp was cleaned by sonication (Branson) for 20 min and washed in 50% ethanol solution before drying under nitrogen flow followed by plasma cleaning for 2 min (Harrick Plasma). Last, the PDMS stamp micropattern areas were soaked in different ECM solutions [laminin 521 (50 μg/ml; Biolamina, LN521-05), fibronectin (Corning, 356008), or collagen IV (Corning, 354233) in phosphate-buffered saline (PBS)] and left to set for 1 hour at room temperature.

After air-drying the ECM proteins atop the PDMS stamp, the excess solution was removed, and the stamp was dried further using nitrogen flow. The freshly prepared hydroxyl-PAAm hydrogel was also dried out by treatment with nitrogen flow. To microprint one ECM, (i) the PDMS stamp was turned so that the patterned surface faced the hydroxyl-PAAm hydrogel surface, (ii) the stamp was gently placed onto the center of the hydroxyl-PAAm hydrogel and left there for 1 hour at room temperature, and lastly, (iii) the PDMS stamp was removed carefully, and the hydrogel was washed three times with PBS to remove unbound proteins. The linkage between the ECM protein and the hydrogel was enabled through HEA integrated into the hydrogel.

For micropatterns with combined ECM proteins, the protocol was the same except that the ECM solutions were added on top of the hydroxyl-PAAm hydrogel and left for 1 hour at room temperature before drying and the subsequent application of the above microprinting protocol. For the removal of laminin in the patterned area, a hydrophobic blank PDMS was microprinted onto the hydrogel and left for 1 hour at room temperature before removal.

### Isolation of Mouse Glomeruli

Animal experiments were approved by the Washington University in St. Louis Institutional Animal Care and Use Committee under protocol number 21-0089. Mouse glomeruli were isolated using an established differential adhesion method with modifications. Briefly, kidneys were collected, minced, and digested in collagenase A (Roche, 10103586001) solution in Hanks' balanced salt solution (HBSS) (1 mg/ml; Gibco, 24020-117) at 37° C. for 15 min. The collagenase A digestion was stopped by adding an equal volume of Dulbecco's modified Eagle's medium (DMEM) with 10% fetal bovine serum (FBS). Next, the suspension containing the dissociated kidney fragments was passed through three differently sized cell strainers (100, 70, and then 40 μm, MIDSCI), and the glomeruli-enriched tissue fragments were collected on top of the 40-μm cell strainer. These were then placed onto 10-cm tissue culture dishes (TPP) for 1 to 2 min to allow the tubular segments to adhere before collecting the glomeruli left in suspension. For higher glomerular purity, the adhesion step was repeated twice. Last, the suspension was spun down under 290 g for 5 min, and the glomeruli were resuspended in primary podocyte culture medium and directly used for the downstream applications.

The mouse podocyte culture medium was prepared as reported earlier. Briefly, for 648 ml of podocyte culture medium, we mixed the following: (i) 3T3L1 conditional media (300 ml), which was the supernatant arising from the culture of the 3T3L1 cell line for 3 days at 37° C. in DMEM culture medium (Gibco, 11965-084) containing 10% FBS (Sigma-Aldrich, F7524) and 1% penicillin-streptomycin (Sigma-Aldrich, P4333); (ii) low-glucose DMEM (204 ml); (iii) Ham's F-12; (iv) I-glutamine (102 ml; Lonza); (v) FBS (30 ml); (vi) penicillin-streptomycin (6 ml) (Sigma-Aldrich, P4333); and (vii) insulin-transferrin-selenium liquid media supplement (6 ml) (Invitrogen).

### Isolation and Reseeding of Primary Mouse Podocytes

To reseed primary podocytes without glomeruli, mouse glomeruli were isolated with the assistance of magnetic microbeads. Briefly, 150 pI of Dynabeads M-450 Epoxy (Invitrogen, 01130646) was diluted into 10 ml of HBSS and used to perfuse the mice. After the perfusion, the kidneys were collected, minced, and digested in collagenase A solution in HBSS at 37° C. for 15 min. Collagenase A activity was stopped by adding an equal volume of DMEM with 10% FBS. Next, the kidney suspension containing various nephron fragments including the glomeruli was passed through a 100-μm cell strainer twice and centrifuged at 200 g for 5 min. The pellet was collected and resuspended in 3 ml of HBSS. Last, the glomeruli were collected using a magnet concentrator, washed three times before being suspended in mouse podocyte culture medium, and cultured in 10-mm culture dishes. After 3 days of culture, the glomeruli and spreading podocytes were lifted off the dish using Cellstripper (Corning, 25-056-CI) and passed through a 40-μm cell strainer to filter out the glomeruli. Last, the pure primary podocytes were reseeded for downstream experiments.

### Isolation of Human Glomeruli

Samples were collected by the Kidney Translational Research Center under a protocol approved by the Washington University Institutional Review Board (IRB 766 #201102312). All patients consented to the research. Kidney nephrectomy tissue was minced, passed through a 250-μm metal cell strainer (MIDSCI) using 1×HBSS solution (Gibco, 24020-117), and collected on a 70-μm cell strainer. The glomeruli-rich suspension was spun down at 2000 g (g=9.81 m/s²) for 5 min and rinsed by passing it through the 70-μm strainer using HBSS. Last, the glomeruli on top of the 70-μm cell strainer were rinsed and resuspended in human primary podocyte culture medium, consisting of DMEM/F12 medium (Thermo Fisher Scientific, 11320-033) with 1% (v/v) insulin-transferrin-selenium (Invitrogen, 41400045), 20% (v/v) FBS (Sigma-Aldrich, F7524), and 1% (v/v) penicillin-streptomycin (Sigma-Aldrich, P4333). These were directly used for the downstream applications.

### Glomerular Culture and Podocyte Spreading on Micropatterned Hydrogels

Before culturing the glomeruli, micropatterned hydrogels were moved inside a six-well culture plate and washed thoroughly with HBSS. After the isolation, glomeruli or primary podocytes suspended in podocyte culture medium were placed on the micropatterned hydrogel. For a single hydrogel, ˜100 pI of the glomerular suspension was added and cultured at 37° C., 5% C02, and 95% humidity. Additional podocyte culture medium (2 ml) could be added to the culture after 10 hours of attachment for primary glomeruli and 2 hours for primary podocytes.

### Culture and Differentiation of Immortalized Cells

IMPs were cultured on the basis of established methods. Briefly, cells proliferated under permissible conditions using RPMI medium (Gibco, 11-875-085) with 10% of FBS in the presence of recombinant mouse interferon-γ (IFN-γ) (10 U/ml; BioLegend, 575304) at 33° C. For differentiation, the medium was replaced with VRAD medium [DMEM/F-12 medium (Gibco, 11-330-032) complemented with vitamin D(3), retinoic acid, and dexamethasone] without IFN-γ, and podocytes were incubated at 37° C. for 7 days. Cells under these nonpermissible conditions stop proliferating, increase their size, and grow processes during the differentiation process. Last, cells were trypsinized and seeded on micropatterned hydrogels. Immortalized 3T3 fibroblasts were cultured using DMEM supplemented with 10% FBS and 1% of penicillin-streptomycin.

### Immunofluorescence Staining

After spreading, cultured podocytes on the hydrogels or glass were fixed in 4% paraformaldehyde (Electron Microscopy Sciences, 15712) for 10 min at room temperature followed by washing with 1×PBS, three times for 6 min each, and then permeabilized using 0.05% Triton X-100 for 10 min at room temperature. Next, samples were blocked using 2% bovine serum albumin (Sigma-Aldrich, A7906) for 30 min at room temperature before applying the primary antibodies overnight at 4° C. Next, samples were washed with 1×PBS, three times for 6 min each before incubating them with the secondary antibodies in PBS for 1 hour at room temperature. Last, the samples were washed in 1×PBS, three times for 6 min, and prepared for mounting using Invitrogen Slow Fade mounting medium (Invitrogen, S36917). The cover glass holding the sample (hydrogel and cells) was bonded to a second cover glass using nail polish. The primary antibodies used were myosin IIA (BioLegend, 909801, rabbit anti-mouse), myosin IIA (Abnova, clone 3C7, mouse anti-human, NH2-terminus), synaptopodin (Synaptic Systems, 163004, guinea pig anti-mouse), laminin α5 [clone 4C7, mouse anti-human], fibronectin (Sigma-Aldrich, F3648, rabbit anti-human), collagen IV (SouthernBiotech, 1340-01, goat anti-human), nephrin (R&D Systems, AF3159, goat anti-mouse), and α-SMA (Invitrogen, 14-9760-82). Alexa Fluor 555 phalloidin (Thermo Fisher Scientific, A34055) was used for actin staining. For mouse myosin antibodies (clone 3C7), samples were antigen retrieved in TE (Tris-EDTA) buffer (pH 9.0) at 65° C. for 4 hours before the permeabilization step.

### Confocal Microscopy

Imaging was performed on a Zeiss LSM 880 confocal microscope [equipped with a unique scan head incorporating a high-resolution galvo scanner along with two photomultiplier tubes (PMTs) and a 32-element spectral detector as well as a transmitted light PMT for differential interference contrast imaging] or a Nikon spinning disk confocal microscope (equipped with a Yokagawa CSU-X1 variable speed Nipkow spinning disk scan head, Andor Zyla sCMOS cameras, and a light-emitting diode-based DMD (Deformable Mirror Device) system for ultrafast photostimulation). Images were taken using a 10× objective for micropatterns with a single protein and using a 60× or 100× oil-immersive objective for other images.

Myosin II Inhibition with Blebbistatin

Blebbistatin (EMD Millipore, 203389-5) was used to inhibit the function of myosin II in the podocytes. On the second day of primary podocyte culture, blebbistatin (50 mM stock solution) was added directly into the podocyte culture medium for a final concentration ranging from 2 to 100 μM. After 2 hours of treatment, the samples were washed with PBS and immediately fixed and stained as mentioned above.

Strain Mapping with Microbead-Embedded Hydrogels

Fluorescent microbeads with a diameter of 0.2 μm (Invitrogen, F8801) were added to the hydrogel's pre-cross-linking solution with a 1:60 dilution ratio before the cross-linking step. After solidification, hydrogels were used for the strain mapping as reported earlier. Briefly, primary mouse podocytes were isolated as described above, seeded on the hydrogels, and allowed to spread for 24 hours. Next, the cells were transferred to a live-imaging chamber before adding CytoD to the culture medium (CytoD, Sigma-Aldrich, 22144-77-0; final concentration, 20 μM). In the final step, the culture medium was changed to 10% SDS to remove the attached cells completely. Cell relaxation and microbead movements were recorded immediately after adding CytoD or 10% SDS, and the strain field was calculated using a strain mapping algorithm that was developed in our previous study.

### Numerical Simulations

A finite element model of a cell process contracting isotropically atop an elastic substrate was studied. The cell and the substrate were approximated as isotropic continua. The cell had an elastic modulus of 1.0 kPa and a Poisson ratio of 0.3. The substrate had an elastic modulus that varied from 0.2 to 6.0 kPa and a Poisson ratio of 0.3. The cell process was given the dimensions shown in FIG. 4C in which the cell shape was assumed to be half of a cylinder capped with a quarter of a sphere, both with a radius of 4 μm. The underlying substrate was 100 μm in width and length and 10 μm in thickness. The substrate base was fixed, and the sides were traction free. Convergence was achieved when the substrate was discretized with 67,354 quadratic elements and the cell with 50,573 quadratic elements. The cell's reference configuration contracted by 10% in the x direction, 8% in the y direction, and 2% in the z direction, considering that the main contractility should follow the x direction (FIG. 3A). This contraction was resisted by elastic stresses stored in the cell and the substrate. In the simulation, different contraction parameters were used. Contraction in all the directions was decreased to 50 or 20% of their original value for medium- and low-contraction situations, respectively. Simulations were performed using Comsol (Comsol Inc., Burlington, MA).

### Quantitative Microscopy of Sarcomeres

Sarcomeric structures were measured using ImageJ (v1.53e with Bio-Formats plugin). Briefly, fluorescence intensity maps were acquired by plotting the intensity along a line drawn perpendicular to the sarcomere structure. For the histogram, the spacing of each sarcomere is calculated by recording the peak location in the intensity map, and n for each group represents the total sarcomere number calculated in that group. For averaged sarcomeric spacing, the mean values were calculated for each intensity map (i.e., each stress fiber that is no less than 6 μm long), while n for each group represents the number of stress fibers analyzed in that group.

### Cell Counting and Scoring

For counting and scoring cell phenotypes, imaged podocytes were divided into several categories, including (i) cells with SLSs centrally and in the periphery, (ii) cells with SLSs only in the periphery, (iii) cells with loose striated patterns of synaptopodin and myosin IIA instead of a mat of SLSs (i.e., widely spaced SLSs), and, lastly, (iv) cells without SLSs. For measuring cell length, the longest axis of the cell was determined (mostly along the micropattern direction), and the length was determined through ImageJ (v1.53e with Bio-Formats plugin).

### Statistical Analysis

Significance analysis for sarcomeric spacing was performed using one-way analysis of variance (ANOVA) followed by Tukey's multiple comparisons test with the GraphPad Prism version 9.0 (GraphPad Software, San Diego, CA). Significance analysis for detachment assay was performed using two-way ANOVA followed by Šidák's multiple comparisons test with GraphPad Prism version 9.0. For all data, differences were represented by a P value that was available upon each of the analyzed data. Data were expressed with each data point when available and together with means or means±SD.

What is claimed is:

1. A podocyte culture system comprising an ECM-patterned substrate, the ECM-patterned substrate comprising:
a compliant substrate, the compliant substrate comprising a hydrogel with a predetermined stiffness ranging from about 0.1 kPa to about 10 kPa; and
a plurality of patterned elements deposited over an exposed surface of the compliant substrate, wherein at least a portion of the patterned elements comprise at least one extracellular matrix (ECM) protein;
wherein the each of the at least one ECM proteins is independently representative of a healthy glomerular basement membrane (GBM) or an abnormal GBM.

2. (canceled)

3. The system of claim 1, wherein the hydrogel comprises a polyacrylamide (PAAm) hydrogel or a hydroxyl PAAM hydrogel.

4. The system of claim 3, wherein the compliant substrate further comprises a protein layer deposited over the exposed surface, wherein the protein layer comprises at least one ECM protein and the plurality of patterned elements are deposited over the protein layer.

5. The system of claim 4, wherein the at least one ECM protein is selected from a laminin, a collagen, a fibronectin, and any combination thereof.

6. The system of claim 5, wherein the at least one ECM protein is selected from a human laminin α5β2γ1 trimer (Lam-521), a fibronectin, a collagen IV, and any combination thereof, wherein the Lam-521 and the collagen IV are representative of the healthy GBM; and the fibronectin is representative of the abnormal GBM.

7. (canceled)

8. The system of claim 3, wherein the compliant substrate comprises a polyacrylamide (PAAm) hydrogel and the plurality of patterned elements comprises Lam-521.

9. The system of claim 4, wherein:
the compliant substrate comprises a polyacrylamide (PAAm) hydrogel;
the protein layer comprises collagen IV; and
the plurality of patterned elements comprises Lam-521;
wherein the system is representative of the healthy GBM.

10. The system of claim 4, wherein:
the compliant substrate comprises a polyacrylamide (PAAm) hydrogel;
the protein layer comprises fibronectin; and
the plurality of patterned elements comprises Lam-521;
wherein the system is representative of the abnormal GBM.

11. The system of claim 4, wherein the predetermined stiffness of the compliant substrate ranges from about 0.2 kPa to about 0.9 kPa, representative of the healthy GBM; or is about 6 kPa, representative of a pathological GBM stiffness.

12. (canceled)

13. (canceled)

14. The system of claim 1, wherein a spacing of adjacent patterned elements of the plurality of patterned elements ranges from about 1 micron to about 20 microns.

15. The system of claim 1, further comprising a plurality of the ECM-patterned substrates and a 96 well-plate, wherein each ECM-patterned substrate is positioned in a well of the 96 well-plate.

16. A method of growing podocytes, comprising:
providing a plurality of podocytes isolated from a biological sample, wherein the biological sample is selected from a urine sample, a kidney biopsy sample, and a glomerular tissue sample;
providing a podocyte culture system comprising:
a compliant substrate, the compliant substrate comprising a hydrogel with a predetermined stiffness ranging from about 0.1 kPa to about 10 kPa; and
a plurality of patterned elements deposited over an exposed surface of the compliant substrate, wherein at least a portion of the patterned elements comprise at least one extracellular matrix (ECM) protein;
wherein the each of the at least one ECM proteins is independently representative of a healthy glomerular basement membrane (GBM) or an abnormal GBM; and
seeding the podocytes onto the ECM-patterned substrate or adjacent to the ECM-patterned substrate.

17. (canceled)

18. (canceled)

19. A method of screening a candidate therapeutic agent, comprising:
providing a plurality of podocytes, wherein the plurality of podocytes are:
isolated from a biological sample of a subject, wherein:
the biological sample is selected from a urine sample, a kidney biopsy sample, and a glomerular tissue sample; or grown out of iPSC-derived kidney organoids; and
the subject is selected from a healthy subject or a subject having, suspected of having, or at risk for having a kidney disease or a glomerular disease;
providing a podocyte culture system comprising:
a compliant substrate, the compliant substrate comprisinq a hydrogel with a predetermined stiffness ranginq from about 0.1 kPa to about 10 kPa; and
a plurality of patterned elements deposited over an exposed surface of the compliant substrate, wherein at least a portion of the patterned elements comprise at least one extracellular matrix (ECM) protein;
wherein each of the at least one ECM proteins is independently representative of a healthy glomerular basement membrane (GBM) or an abnormal GBM;
culturing the plurality of podocytes using the podocyte culture system;
contacting the plurality of podocytes with the candidate therapeutic agent; and
observing an effect of the candidate therapeutic agent on correcting cellular homeostasis.

20. The method of claim 19, wherein the method is a patient-specific method, wherein the method further comprises providing the biological sample from a selected patient and the method is a patient-specific method.

21. (canceled)

22. (canceled)

23. The method of claim 19, further comprising detecting sarcomere-like structures (SLSs), synaptopodin in SLSs, or motor protein myosin IIA in SLSs as markers of health or injury of the plurality of podocytes.

24. The method of claim 23, further comprising observing the growth of the plurality of podocytes or the markers of health or injury of the plurality of podocytes in response to the candidate therapeutic agent, wherein the markers of health or disease comprise migration or biological markers selected from SLSs, synaptopodin, α-actinin 4, myosin IIA, and any combination thereof.

25. (canceled)

26. (canceled)

27. (canceled)

28. (canceled)

29. The method of claim 19, wherein observing the effect of the candidate therapeutic agent on correcting cellular homeostasis further comprises observing or identifying changes in podocyte cellular and cytoskeletal patterns or observing changes in cellular homeostasis.

30. The method of claim 19, wherein the plurality of podocytes cultured using the podocyte culture system upregulates sarcomere-like structures.

31. The method of claim 19, wherein the ECM-patterned substrate is further coated or printed with an antibody to a membrane protein, or to a binding partner of a membrane ligand.

32. The method of claim 31, wherein the ECM-patterned substrate is further coated or printed with an antibody to a membrane protein comprising nephrin.

33. (canceled)
